(19) United States
(12) Patent Application Publication
(10) Pub. No.: US 2006/0046277 A1
(43) Pub. Date: Mar. 2, 2006

(54) Protein kinase and phosphatase substrates and multiplex assays for identifying their activities

(76) Inventors: Alexander Sergeevich Belyaev, San Diego, CA (US); Angelo Stephen Kolokithas, La Mesa, CA (US); Craig Robert Monell, La Jolla, CA (US)

Correspondence Address: MOAZZAM LATIMER LLP, 1474 NORTH POINT VILLAGE CENTER #320, RESTON, VA, 20194-1190, US

(21) Appl. No.: 10/931,251

(22) Filed: Sep. 1, 2004

(51) Int. Cl. C12Q 1/48; C12N 9/12

(52) U.S. Cl. 435/15; 435/194

(57) ABSTRACT

The present invention provides protein kinase and protein phosphatase substrates, methods of detecting protein kinases and protein phosphatases, and kits for detection of protein kinases and protein phosphatases. The substrates, methods, and kits use multiple substrates that can easily be differentiated from all other substrates, thus enabling rapid and easy detection of protein kinase or protein phosphatase activities. The invention also provides methods of directionally cloning nucleic acids.

### BACKGROUND OF THE INVENTION

1. Field of the Invention

The present invention relates to assays for the presence, level of activity, or both, of protein kinase and protein phosphatase enzymes in a sample or living cell. In particular, the present invention relates to compositions and assays for detection of one or more specific protein kinase enzymes or one or more protein phosphatase enzymes in a sample or cell, where the sample or cell comprises one or more protein kinases or one or more protein phosphatases, and where the compositions and assays comprise different, uniquely identifiable substrates for each specific protein kinase or phosphatase to be analyzed.

2. Description of Related Art

A major goal of many research programs focusing on cell physiology in both normal or healthy cells and in cells associated with a disease or disorder is to identify proteins that are active under various conditions and at various times during the cell cycle, under various environmental conditions, and during various times of progression of a disease or disorder. Understanding the complement of proteins that are active under particular conditions or at particular times can provide a researcher with information about the proteins that are involved, either through their presence and activity level or through their absence, in a particular physiological response. Among the physiological responses that can be monitored are production of toxins, change in cell surface protein make-up, production of immune system-specific proteins, initiation or cessation of cell replication or division, and programed cell death. Likewise, an understanding of the complement of active proteins in a given cell at a given time or under a given condition can provide the researcher with information on which proteins are involved in a disease or disorder. It can also identify proteins that are involved in initiating, monitoring, sustaining, and halting biological responses, such as activation cascades, that are necessary for cells to respond to stimuli.

Due to their physiological relevance, variety, and ubiquitousness, protein kinases have become one of the most studied enzymes in both commercial and academic settings. Wide interest in protein kinases is well justified, as they are key regulators of many cell functions, including signal transduction, transcriptional regulation, cell motility, cell division, and oncogenesis. Indeed, a major focus of recent research is on kinases that might inhibit cancer growth or promote cancer cell death. Therefore, technologies that permit researchers to detect the presence of kinases and the levels of specific kinase activities in cell lysates as well as in live cells are extremely important for cancer research. In addition, because protein kinases are known to be involved in numerous cellular functions, technologies that permit researchers to detect the presence and level of activities of specific protein kinases are important for the study of numerous other diseases and disorders.

In recent years, various technologies for testing protein kinase activities have been developed. However, most of the advances have been made in the area of homogenous assays amenable to a high-throughput screening (HTS) format. Thus, most of the recent advances are directed to formats that are typically used by researchers at large commercial labs, such as by researches at pharmaceutical companies engaged in discovery of potential drug candidates. Examples of kinase detection technologies available for HTS include the scintillation proximity assay (commercially available from Amersham, Piscataway, N.J.), fluorescent polarization (commercially available from Perkin Elmer, Wellesley, M A; Invitrogen, Carlsbad, Calif.; Panvera, Madison, Wis.; and Molecular Devices, Sunnyvale, Calif.), the LANCE™ (Perkin Elmer, Wellesley, M A) assay based on time-resolved fluorescence transfer and time-resolved fluorescence, fluorescence quenching (commercially available from Pierce, Rockford, Ill.), the Z-LYTE™ (Invitrogen/Panvera, Carlsbad, Calif.) assay based on different sensitivity of phosphorylated and non-phosphorylated peptides to proteolytic cleavage, the AlphaScreen™ (Perkin Elmer, Wellesley, M A) assay, which is a bead-based non-radioactive luminescent proximity assay, Kinase-Glo™ (Promega, Madison, Wis.), which is a luminescent multi-well assay based on depletion of ATP in a reaction mixture, and an electrocapture assay commercially available from Nanogen (San Diego, Calif.), which is based on change in the charge of a peptide after phosphorylation.

Although much of the recent effort in development of protein kinase assays has been focused on the HTS format, products are also available for low-throughput analysis of protein kinase activities. Such assays are appealing for companies and laboratories that typically do not have specialized equipment for HTS, or that typically investigate a limited number of protein kinase activities in a single assay. For example, SpinZyme™ kits (Pierce, Rockford, Ill.) and the PepTag® assay (Promega, Madison, Wis.) allow testing for only protein kinase A (PKA) or protein kinase C (PKC) activities. Although these types of products can be useful in certain situations, products limited to testing 1-2 activities are not very informative for elucidating the activation of numerous different pathways. The same is generally true for assays based on myelin basic protein (MBP), which is a substrate that can be indiscriminately phosphorylated by many Ser/Thr kinases. Such technologies are useful for discovery of inhibitors of a particular kinase used in the assay with MBP; however, they cannot be applied to a complex protein mixture containing many kinases. Technologies based on MBP include an ELISA-based kit from Upstate Biotechnology (Waltham, Mass.) and the LabMAP™ system (Luminex, Austin, Tex.).

Protein kinase substrates (i.e., peptide sequences to which protein kinases specifically bind) are important tools for investigation of protein kinase activities, such as signal transduction mechanisms, including cell responses to specific stimuli. Various protein kinase substrates are commercially available. For example, Upstate Biotechnology (Waltham, Mass.) markets short synthetic peptide substrates, which can be specifically phosphorylated by certain protein kinases. The peptide substrates for all of the protein kinases that can be assayed using this technology are all about the same size. Thus, multiple different peptide substrates cannot be conveniently used to detect different protein kinases in a single sample. Furthermore, very few peptide substrates are provided with affinity purification tags, which largely limits their usefulness to characterization of purified protein kinases. That is, the lack of affinity purification tags renders the substrates unsuitable for use in crude cell lysates.

In addition, Jerini (Berlin, Germany) offers peptide arrays containing hundreds of peptide substrates derived from natural protein phosphorylation sites, as well as their artificial variations. It has been reported that such arrays are very useful for characterization of substrate specificity of purified protein kinases (Lizcano, J. M. et al., J. Biol. Chem. 277(31):27839-49, 2002). However, the overwhelming majority of the peptides used in these arrays remain uncharacterized with respect to substrate specificity or cross-reactivity with different kinases, as they simply represent peptide sequences containing a phosphorylation site.

Investigation of protein kinase activities in living cells is considered by many to be as important as, or even more important than, the ability to follow changes in a specific kinase activity in a cell lysate, as intact cells are typically more physiologically relevant. However, current commercial technologies are inadequate to achieve this goal. For example, a widely used technique uses phospho-specific antibodies to monitor kinase activity on endogenous pathway proteins. However, this technique requires processing of the cells to fix them and render them permeable to the antibodies, which could result in loss of target. Likewise, it does not permit multiple direct sampling of a single sample. Use of this system in conjunction with currently available protein kinase substrates would also require delivery of the formed protein kinase substrates to the cells, which is a difficult task to achieve reliably.

Control of activation pathways and activities of various proteins also relies on the presence and activity of protein phosphatases. For example, protein phosphatases are implicated in deactivation of proteins that are phosphorylated by protein kinases, and thus play a role in activating or deactivating various proteins involved in physiological responses to various stimuli. Numerous studies have been performed to identify and characterize such proteins. The studies have identified a number of phosphatases and the specific sequences that they recognize and modify (by dephosphorylation of phospho-amino acids).

Constructs and assays for expressing and characterizing proteins and substrates to which they bind have been reported. For example, U.S. Pat. No. 5,514,581 discloses polymers, and methods of producing them, that comprise multiple repeating units of a sequence of interest taken from structural proteins (e.g., silk fibroin, elastin, collagen, keratin). The repeating units can be interrupted by intervening units having a variety of activities. According to the disclosure, the repeating units function to provide a structure that allows the intervening units to interact with the environment, and thus with substrates. Likewise, U.S. Pat. No. 5,830,713 discloses methods for preparing nucleic acids comprising repetitive sequences, such as those encoding functional motifs (e.g., structural units of elastin, silk fibroin, keratin, collagen). The repetitive sequences can be fused in-frame to heterologous sequences encoding peptides, such as host-cell peptides.

U.S. Pat. No. 6,348,310 and U.S. Pat. No. 6,753,157 disclose a method, a peptide substrate, and kits for quantitating the activity of a selected protein kinase on the peptide substrate. The method comprises conjugating a biotin to a peptide substrate for the kinase, reacting the substrate with a kinase, and detecting a modified substrate. The biotin is used to purify the substrate from the reaction mixture. Each assay is performed with a single substrate and tests for a single kinase.

In addition, U.S. Pat. No. 6,599,711 discloses a method for detecting a protein kinase of interest. The method is based on detection of ATP and ADP levels using a bioluminescence reaction. The methods rely on the use of the intact protein kinase and its intact substrate protein.

In view of the state of the art, it is evident that advances in protein kinase substrates and in assays using them are necessary to provide low-throughput assays that are simple, sensitive, robust, and suitable for assaying multiple protein kinases at one time. Likewise, advances in protein phosphatase substrates and in assays using them are necessary to provide low-throughput assays having similar characteristics.

### SUMMARY OF THE INVENTION

The present invention addresses needs in the art by providing protein kinase and protein phosphatase substrates, compositions and cells comprising those substrates, and assays for detecting the presence and levels of activities of protein kinases and protein phosphatases. In general, the protein kinase and protein phosphatase substrates, compositions, cells, and assays permit detection of multiple different protein kinases and multiple different protein phosphatases in a single sample or cell without the need for complex, time-consuming, or expensive reaction or detection equipment.

The improved substrates of the invention can be cost effective (for an in vitro assay, often less of the substrate of the present invention can be needed compared to substrates that are currently commercially available) and can provide improved sensitivity, which is especially valuable for cell-based assays. This invention can be used to identify protein kinases, sets of protein kinases, protein phosphatases, and sets of protein phosphatases that are active or inactive in numerous different cellular activation states, and can be used to compare the complement of protein kinases or protein phosphatases in cells under different activation states. It thus can be used to identify active and inactive kinases and phosphatase in certain disease states, and thus can identify drugs that can be used to treat those disease states. By identifying protein kinases and protein phosphatases that are active or inactive under various cellular activation states, including certain disease states, the invention can lead to the identification of in vivo substrates of those kinases and phosphatases. Accordingly, it can be used to identify targets for drugs that can be used to treat abnormal activation states, including those of diseases or disorders. The ability to combine different substrates in the same reaction and then separate them afterwards for analysis is a distinct advantage over current technologies. Thus, the present invention provides valuable tools for investigating the effects of potential therapeutics or other factors on a variety of signaling pathways.

In a first aspect, the invention provides protein kinase and protein phosphatase substrates. Each individual protein kinase substrate contains an amino acid sequence that is specifically recognized and/or phosphorylated by a protein kinase or a group of protein kinases that share a common recognition and/or phosphorylation target sequence. The protein phosphatase substrates are identical to the protein kinase substrates in their primary amino acid sequences, but are provided as phosphoproteins that can be dephosphorylated by specific protein phosphatases. The protein kinase and protein phosphatase substrates are fusion polypeptides that comprise not only the recognition and phosphorylation or dephosphorylation site(s), but a tag that facilitates purification of the substrate from the reaction mixture and can also permit secondary detection of the substrate. The protein kinase and phosphatase substrates can include a single recognition/phosphorylation/dephosphorylation site or multiple sites arranged as concatamers of peptides containing the recognition/phosphorylation/dephosphorylation target sequence to permit rapid and easy detection based on size, and to enhance signal strength when labeled phosphate groups are used to phosphorylate the substrates.

In a second aspect, compositions are provided that comprise one or more protein kinase or protein phosphatase substrates of the invention. In general, the compositions comprise one or more substances that are compatible with stable maintenance of the substrates of the invention for a sufficient amount of time for the substrates to be used in an assay, or that are compatible with reaction or detection conditions that are used to detect the presence or activity of a protein kinase or protein phosphatase. The compositions typically, but not necessarily, include a sample or cell that is suspected of containing a protein kinase or protein phosphatase of interest.

In a third aspect, nucleic acids are provided that encode a protein kinase or protein phosphatase substrate of the invention. The nucleic acids generally comprise sequences that encode at least one recognition and/or phosphorylation site for a protein kinase or at least one recognition and/or dephosphorylation site for a protein phosphatase. The nucleic acids can also comprise sequences that encode a tag that facilitates purification of the encoded substrate from a reaction mixture, and that can permit secondary detection of the substrate. Typically, the sequences encoding the tag are fused in-frame with the sequences encoding the substrate in order to produce a fusion protein for use in the assays of the invention.

In a fourth aspect, the invention provides methods of making nucleic acids comprising a sequence encoding a tag fused in-frame to a sequence encoding one or multiple in-frame repeats of a sequence encoding an amino acid sequence of interest. In general, the methods comprise engineering a nucleic acid sequence encoding the amino acid sequence of interest such that one end of the nucleic acid sequence is a restriction endonuclease cleavage site product that can be ligated into a corresponding restriction endonuclease cleavage site to form a competent endonuclease cleavage site. The other end of the nucleic acid sequence is engineered to have an incompetent restriction endonuclease cleavage site product that, while resembling a restriction endonuclease cleavage site, does not form a complete restriction endonuclease cleavage site if ligated to a corresponding cleavage site product. The nucleic acid is engineered such that association and ligation of the incompetent site product to a competent site product creates an incompetent site that is resistant to later cleavage by the endonuclease. The nucleic acid is also designed such that the ligation product that creates an incompetent endonuclease site creates an in-frame fusion of two nucleic acids. The nucleic acid design also eliminates association and ligation of two incompetent endonuclease site products. In addition, the nucleic acid is designed such that association and ligation of two competent endonuclease sites (either from two engineered molecules or from an engineered molecule and a vector, etc.) forms a competent site. In the method, multiple copies of the engineered sequence are mixed with a sequence encoding a tag or a nucleic acid vector comprising a sequence encoding a tag, and the various nucleic acids are ligated together. The resulting constructs, the population of which comprises different numbers of engineered molecules inserted into the vector, is then digested with the restriction endonuclease for which the engineered nucleic acids have sites or incompetent sites. Cleavage of the competent sites occurs, leaving only constructs having one or more engineered sequences fused in-frame with each other and with the tag, all. See FIG. 1. If the tag and engineered molecules were ligated in the absence of a vector, the resulting in-frame construct can be digested with the appropriate nucleases (if necessary) and ligated into the vector. In embodiments, the engineered sequences encode target sites or target site units.

In a fifth aspect, the invention provides compositions comprising the nucleic acids of the invention. In general, the compositions comprise one or more substances that are compatible with stable maintenance of the nucleic acids of the invention for a sufficient amount of time for the nucleic acids to be used. The compositions typically, but not necessarily, include an aqueous buffer compatible with other components used to insert nucleic acids into cells, substances present in in vitro transcription assay mixtures, or cellular components present in cells or in cell lysates.

In a sixth aspect, the invention provides cells comprising a protein kinase or protein phosphatase substrate of the invention, a nucleic acid of the invention, or both. The cells of the invention are generally useful for expressing the protein kinase or protein phosphatase substrates of the invention for either purification and later use in in vitro or in vivo assays. They are also generally useful for producing more (i.e., amplifying) nucleic acid of the invention and for maintaining a nucleic acid of the invention.

In a seventh aspect of the invention, assays are provided. The assays are designed to provide information on the presence of one or more protein kinases or one or more protein phosphatases in a sample, such as a cell or cell lysate. They are also designed to provide information on the level of activity of one or more protein kinases or one or more protein phosphatases in a sample. Thus, the assays can identify a single protein kinase activity or protein phosphatase activity, or can identify multiple, different protein kinase or protein phosphatase activities (i.e., a multiplex assay). The assays generally comprise providing at least one protein kinase or protein phosphatase substrate having a known protein kinase binding and/or phosphorylation site (i.e., a protein kinase target site) or a known protein phosphatase binding and/or dephosphorylation site (i.e., a protein phosphatase target site), providing a sample suspected of containing at least one protein kinase or protein phosphatase for which a target site has been defined, combining the substrate and the sample for a sufficient amount of time for phosphorylation of the target site to occur if a suitable protein kinase is present or for dephosphorylation of the target site to occur if a suitable protein phosphatase is present, and determining if a phosphorylated substrate is present. The presence of a phosphorylated substrate in a kinase assay indicates the presence of one or more specific kinases in the sample. The absence of a phosphorylated substrate or the lessening of a signal derived from a phosphorylated substrate in a phosphatase assay indicates the presence of one or more specific phosphatases in the sample.

In a final aspect, the present invention provides kits. Kits of the invention can comprise one or more substrates according to the invention, one or more nucleic acids of the invention, one or more compositions of the invention, or combinations of two or all of these. The kits generally are designed to provide some or all of the components needed to perform an assay according to the invention. Thus, the kits can, but do not necessarily, contain all of the components needed to perform an assay according to the invention.

### BRIEF DESCRIPTION OF THE DRAWINGS

The accompanying drawings, which are incorporated in and constitute a part of this specification, illustrate several exemplary embodiments of the invention and, together with the written description, serve to explain the principles and details of the invention.

FIG. 1 depicts a general scheme for construction of plasmid vectors for expression of protein kinase and protein phosphatase substrates according to one embodiment of the invention.

FIG. 2 depicts a general scheme for increasing the number of monomers in a concatamer encoding a protein kinase or protein phosphatase substrate according to one embodiment of the invention.

FIG. 3 depicts a general scheme for construction of plasmid vectors expressing GST-stuffing fragment fusions and for combining them with protein kinase and protein phosphatase substrates.

FIG. 4 depicts recovery of a soluble protein kinase substrate ladder from crude cell extracts.

FIG. 5 depicts an SDS-PAGE gel of protein kinase substrates.

FIG. 6 depicts phosphorylation of various protein kinase substrates.

FIG. 7 shows that an increase in substrate reactivity can be contingent upon the number of the substrate peptide repeats in GST fusion proteins.

FIG. 8 shows the results of a Bradford assay performed with soluble proteins and the same proteins attached to beads.

FIG. 9 shows that the band intensity on Coomassie Blue stained gels of soluble and on-the beads protein preparations are similar when adjusted to the same concentration based on the Bradford assay.

FIG. 10 shows that repeated freeze-thaw cycles do not significantly alter the structure or binding capacity of beads comprising substrates of the invention. Panel A—beads before freeze/thaw cycles; Panel B—supernatant before freeze/thaw cycles; Panel C—beads after 5 freeze/thaw cycles; Panel D—supernatant after 5 freeze/thaw cycles.

FIG. 11 shows an SDS-PAGE gel of the GST-IKK×4 substrates after treatment as depicted in FIG. 10.

FIG. 12 shows that the phosphorylation of a 4-member protein kinase ladder with protein kinases specific to the ladder components can be easily detected using standard protein gel techniques.

### DETAILED DESCRIPTION OF EMBODIMENTS OF THE INVENTION

Reference will now be made in detail to various exemplary embodiments of the invention. The description is provided to aid in explaining certain details of the invention, and should not be construed as limiting the scope of the claims to the particular details described herein. Unless otherwise indicated, terms used herein are used in accordance with their ordinary and customary meaning within the biotechnology (including protein biochemistry) field at the time of the invention, as will be evident by the context in which they are used, it being understood that various terms may have different meanings in the biotechnology field than in the general English language or in other scientific fields.

In a first aspect, the invention provides protein kinase and protein phosphatase substrates. According to the present invention, a protein kinase substrate is a peptide, polypeptide, or protein (the three terms being used interchangeably from this point on) that contains at least one sequence of amino acids (i.e., a site) that is recognized by at least one protein kinase, and that contains at least one sequence of amino acids (i.e., a site) that is phosphorylated by the protein kinase. The recognition site and the phosphorylation site may contain the same residues, may contain overlapping residues, or may be separate sites within the substrate. Because the amino acids that are important in protein kinase recognition sites and phosphorylation sites are typically within five residues of each other along the primary amino acid sequence, for ease of reference, the recognition site and phosphorylation site will be, from this point on, referred to as a “target site” (in the singular), even where the particular substrate contains a recognition site that is distinct and separated from the phosphorylation site. Target sites are provided based on known binding and/or phosphorylation sites for known protein kinases. Thus, identification of target sites and design of substrates that are specific for a particular protein kinase or group of protein kinases is well within the skill of those of skill in the art, and does not require undue experimentation.

According to the present invention, protein phosphatase substrates are identical to the protein kinase substrates in their primary amino acid sequences, but are provided as phosphoproteins that can be dephosphorylated by specific protein phosphatases. Protein phosphatase recognize specific phosphoprotein sequences and cleave the phosphate group from the protein sequence. Different phosphatases recognize different phosphoprotein sequences, and are thus specific for certain target sequences (although some phosphatases recognize the same or overlapping phosphoprotein sequences). For the purpose of this invention, unless otherwise specifically noted, the target site defined for the protein kinase that specifically phosphorylates a substrate is considered to be the target site for the phosphatase that specifically dephosphorylates the substrate.

The protein kinase and protein phosphatase substrates can include a single target site, which can be phosphorylated by a known protein kinase if present in the sample being tested, such phosphorylation permitting detection of the substrate by way of radioactive labeling, creation of a site recognized by a anti-phospho-peptide antibody, or other techniques known to those of skill in the art. Likewise, the target site can be dephosphorylated by a known protein phosphatase if present in the sample being tested, such dephosphorylation permitting detection of the substrate.

Alternatively, the substrate can include more than one (e.g., two, three, four, five, six, seven, eight, nine, ten, or more) target site, each of which including a target site for the same protein kinase or protein phosphatase. In embodiments, the substrate comprises two or more of the same target site arranged as repeats along the primary amino acid sequence. In essence, such a substrate comprises concatamers of the target site. Of course, the target site can be present on a peptide having non-target sequences flanking one or both sides of the target site, the entire peptide serving as a target site unit. Among other things, providing such flanking sequences can aid in phosphorylation or dephosphorylation of the substrate, aid in solubility of the substrate, and aid in purifying or analyzing the substrate. Likewise, the target sequence can be fused to a spacer sequence or between two spacer sequences to achieve the same effect. Numerous types of such flanking sequences and spacers and numerous ways of introducing such flanking sequences or spacers are known to those of skill in the art, and any suitable one or combination of more than one can be used to insert one or more non-target sequence residues into the substrates of the invention.

As will be described in more detail below, providing multiple repeats of the target sequence permits one to easily design substrates that are specific for a given protein kinase (or group of kinases sharing a common target sequence) or a given protein phosphatase (or group of phosphatases sharing a common target sequence). It also permits one to easily design substrates that have a molecular weight that can provide easy, rapid, and unequivocal identification of the substrate at the completion of an assay. In addition, providing multiple target sites can increase the number of phosphorylation or dephosphorylation events per substrate, thus increasing the amount of label introduced (for a kinase reaction) or removed (for a phosphatase reaction) per substrate and increasing the sensitivity of the substrates and assays.

The substrates of the invention also comprise a tag fused to the target site sequence(s) or target site units. The tag is used to isolate the substrates of the invention after exposure to the sample suspected of containing a protein kinase or protein phosphatase. The tag may also be used to identify a substrate according to the invention. Typically, when used to identify a substrate, the tag is used as a secondary or confirmatory identification marker, to confirm that the peptide identified by the primary screening method is a substrate according to the invention, and not an unrelated, non-specific contaminant. In embodiments, the tag can be fused to the target site(s) or target site unit(s) in such a way that it can be cleaved, for example by proteolytic enzymes, from the target site(s) or target site unit(s). Release of the tag from the rest of the substrate can be advantageous in detecting substrates with different numbers of concatamers or in detecting phosphorylated substrate products when the tag itself has undergone specific or non-specific phosphorylation during exposure to the sample suspected of containing a protein kinase. However, such cleavage is not always necessary because the methods of the invention permit one to determine whether phosphorylation of the tag has occurred.

The invention envisions the use of any suitable tag that can be covalently bound to a peptide. In embodiments, a protein tag is used. Typically when a protein tag is used, it is fused directly to the target site(s) or target site unit(s) through a peptide bond, although a linker (also referred to herein as a stuffer) can be included between the tag and the target site(s) or target site unit(s). Such a linker can be any molecule that is capable of linking two peptides. Peptide linkers are preferred. The linker can be of any length or configuration as long as it does not impair the ability of a protein kinase or protein phosphatase to access one or more target sites on the substrate. An exemplary tag is the glutathione-S-transferase (GST) protein, which can be fused to target site(s) or target site unit(s) and used to affinity purify the substrates to glutathione-containing solid supports. Other suitable tags are known in the art and include, but are not limited to, biotin, bovine serum albumin, hexahistidine, and the like. Substrates comprising tags can be used both in solution (with or without later affinity purification by binding to a solid support) and bound to a solid support. As used herein, solid support means any substance that provides a solid surface on which the substrates of the invention can be bound, either directly or through a tag. Any known solid support known in the art can be used, including, but not limited to, beads (latex, plastic, glass, etc.), plates (plastic, glass, etc.), membranes (nylon, plastic, polysaccharide, etc.), and the like.

In a second aspect, compositions are provided that comprise one or more protein kinase or protein phosphatase substrates of the invention. The amount of substrate(s) in a composition can be adjusted based on numerous considerations, including, but not limited to, the amount of protein kinase or protein phosphatase suspected of being in a sample to be assayed. One of skill in the art can adjust the amount of substrate(s) without undue experimentation. For example, a substrate can be present in a composition in an amount of up to or about 0.1 μg, about 0.2 μg, about 0.25 μg, about 0.3 μg, about 0.4 μg, about 0.5 μg, or more. Compositions comprising more than one substrate can include the same or different amounts of each, each substrate being present at any suitable amount.

In embodiments, multiple substrates are provided in a composition, each individual protein kinase substrate being specific for the same protein kinase or group of protein kinases that share a common target site, and each protein phosphatase substrate being specific for the same protein phosphatase or group of protein phosphatases that share a common target site. As discussed above, each substrate is designed to be specific for a particular protein kinase or group of protein kinases, or each protein phosphatase or group of phosphatases, based on the presence of at least one target site to which the protein kinase(s) or protein phosphatase(s) specifically bind and/or phosphorylate/dephosphorylate. The invention contemplates the use of one or multiple target sites on a substrate. Thus, in embodiments, compositions are provided in which multiple substrates are provided, each with target sites that are specific for a particular protein kinase or group of protein kinases (or particular protein phosphatase or group of protein phosphatases), but each having a different number of repeats of the target site (or target site unit). The relative amounts of each substrate (i.e., total number of copies of each substrate provided in the composition) can be selected by the practitioner to provide various advantages. For example, because substrates having increasing numbers of repeats of the target site generally are phosphorylated to a greater extent than substrates with fewer repeats, one might wish to include more copies of substrates with relatively few repeats of the target site in order to obtain a detectable signal (e.g., radioactive emission from a radioactive phosphorus) that is similar in intensity to that obtained from substrates with relatively many target sites. Similar considerations can be taken into account when determining the relative amounts of phosphatase substrates to include in compositions.

Providing substrates that are specific for a single protein kinase (or multiple protein kinases that specifically bind to the same target site) or protein phosphatase (or multiple protein phosphatases that specifically bind to the same target site), but that vary in the number of target site repeats results in a characteristic pattern of detectable, labeled substrates after specific interaction with a protein kinase, or a characteristic pattern of detectable loss of labeled substrates after interaction with a protein phosphatase. For example, if one were to make three substrates having successively longer concatamers of a target site unit of 20 amino acid residues (by one target site unit each time), then by contacting a composition comprising the resulting substrates with a sample containing a protein kinase that specifically binds to the target site, one would obtain three labeled products that were approximately two kilodaltons (kDa) in size different (i.e., products of 2, 4, and 6 kDa, if the tag of the substrate were cleaved prior to assaying for labeled substrate). If one chose to maintain the tag on the substrate while detecting labeled substrate, the size of the tag would be added to the size of the target site or target site unit, and one would look to the appropriate size range to identify the presence of a labeled substrate.

In embodiments, multiple different substrates (or sets of substrates having differing numbers of repeats of the same target site or target site unit) are provided in a composition, each different substrate being specific for a different protein kinase or group of protein kinases, or a different protein phosphatase or group of protein phosphatases. Such compositions can be used to identify the presence, and preferably the activity, of multiple different protein kinases or protein phosphatases in a single sample to be tested. Each substrate or set of substrates that are specific for one protein kinase are designed to provide a unique characteristic for detection, thus enabling one to rapidly and confidently identify the presence of a particular protein kinase in a sample being tested. Likewise, each substrate or set of substrates that are specific for one protein phosphatase are designed to provide a unique characteristic for detection, thus enabling one to rapidly and confidently identify the presence of a particular protein phosphatase in a sample being tested.

More specifically, each substrate (or set of substrates) that is specific for a given protein kinase or a given protein phosphatase has a characteristic that is different from all other substrates (or sets of substrates), such that detection of that characteristic indicates the presence in the tested sample of a protein kinase or protein phosphatase that specifically binds and phosphorylates (in the case of a kinase) or binds and dephosphorylates (in the case of a phosphatase) the particular target site of the substrate. For example, each substrate that is specific for a given protein kinase can be tagged with the same tag (e.g., GST), which is different from the tags used in conjunction with the substrates for each other protein kinase (e.g., biotin, hexahistidine, etc.). Detection of a labeled substrate product with a given tag indicates the presence in the tested sample of a protein kinase or protein phosphatase that is specific for the target site on the substrate associated with that tag. Because the target sites are designed based on known target sites for known protein kinases and known protein phosphatases, the identify of active protein kinases and active protein phosphatases can be determined based on detection of the tag. If multiple protein kinases or multiple protein phosphatases are specific for the same target site, then the results indicate that at least one of the kinases or at least one of the phosphatases is present in the tested sample.

In embodiments, the tag is the same for all substrates present in the composition, regardless of the protein kinase or protein phosphatase for which they are specific. Thus, in these embodiments, use of the tag to differentiate the types of protein kinases or protein phosphatases present in a sample will not be adequate (although it can be used to confirm that a detected labeled substrate is, in fact, a substrate and not a non-specific protein). To identify the presence (and preferably activity) of protein kinase(s) or protein phosphatase(s) in a sample in these embodiments, the sizes of the target sites are varied in a controlled and predetermined manner. More specifically, the size and number of target site units on a particular substrate are designed to provide a substrate having a known molecular weight. Each specific substrate is designed to have a molecular weight that differs from other substrates to be used in the composition. Thus, each substrate has a unique molecular weight within the composition. Upon contact with a sample containing a protein kinase (or group of kinases) that specifically binds and phosphorylates a given target site, a labeled substrate is produced. This labeled substrate can be detected and, based on its unique size, can be identified. Identification of the labeled substrate indicates the presence of a particular protein kinase in the sample tested. When multiple protein kinases are specific for a given target site, the results indicate the presence of at least one of those protein kinases in the sample. In a similar manner, upon contact with a sample containing a protein phosphatase (or group of phosphatases) that specifically binds and dephosphorylates a given target site, the amount of labeled substrate is decreased. The loss in labeled substrate can be detected and, based on the unique size, the substrate can be identified. Identification of diminished (or absent) substrate indicates the presence of a particular protein phosphatase in the sample. When multiple protein phosphatases are specific for a given target site, the results indicate the presence of at least one of those protein phosphatases in the sample.

To provide even further confidence that a particular protein kinase or protein phosphatase is present in the tested sample, one can include multiple substrates that are specific for a given protein kinase or phosphatase, each such substrate having a different molecular weight due to the presence of different numbers of repeats of the target site or target site unit. In other words, one can provide a set of specific substrates, each particular substrate having a known molecular weight. When a protein kinase that specifically interacts with the target site(s) present on the substrates is present in the sample being tested, a characteristic pattern of labeled substrate products is produced, each individual member substrate having a characteristic molecular weight. In essence, a ladder of labeled substrates is produced, the ladder being made of multiple different labeled substrates of characteristic sizes, based on the pre-selected number of concatamers of target site or target site units. Likewise, when a protein phosphatase that specifically interacts with the target site(s) present on the substrates is present in the sample being tested, the characteristic pattern of labeled substrates that were provided in the composition (each having a different molecular weight) is altered by reduction or elimination of one or more substrates. In essence, the ladder of labeled substrates (the ladder being characteristic for a given target site) is reduced in signal intensity or lost.

The compositions of the invention are thus useful for identifying the presence of multiple protein kinases or multiple protein phosphatases in a single sample, but without the need for complex or expensive machinery. The ability to rapidly and easily detect the presence of multiple different protein kinases or phosphatases in a sample also allows one to identify multiple (or all) of the members of a given cell signaling pathways, using crude cell lysates or whole cells in which the various substrates are expressed.

In exemplary embodiments, each substrate comprises a GST tag. The GST tag, like any other tag according to the invention, permits binding of the substrates to a solid support (in this case, a glutathione-containing bead), and thus permits identification of various protein kinases or phosphatases in an assay format that includes the bound substrates or in an assay format that includes phosphorylation or dephosphorylation of substrate in solution, followed by purification of labeled substrate through GST-glutathione bead interaction. A specific exemplary embodiment, which is detailed in the examples below, provides a wide range of protein kinase substrates to investigate a variety of protein kinase activities in cell extracts. In this embodiment, the substrates contain a Schistosoma japonica Glutathione-S-Transferase (GST) tag to facilitate their purification from other endogenous phosphorylated proteins present in the extracts. The substrates range in size from about 28 to about 78 kDa, to allow their use in a multiplex format, i.e., composing a mixture of several substrates (substrate ladder), and identification of individual substrates in the mixture according to their characteristic mobilities in SDS-PAGE. In this embodiment, most of the substrates contain concatamers of the target site sequence, which improves their sensitivity.

While it is envisioned that compositions comprising substrates that are specific for all known protein kinases or protein phosphatases can be created, it is likely that practitioners will want to select only those substrates that are relevant for protein kinases or protein phosphatases suspected of being present in a sample to be tested. Doing so will reduce the cost and complexity of the assay and will permit the practitioner to select characteristics (e.g., molecular weights) for the substrates that are convenient and easy to differentiate from the other substrates in the composition. For example, protein kinase substrates can be selected to be specific for the most widely used and well characterized protein kinases, such as those involved in major signal transduction pathways. Though any particular assay reaction composition will typically contain fewer probes than the number of probes used in some commercially available protein arrays, a carefully selected set of substrates according to the present invention can provide comparable information to such protein arrays for the major pathways targeted. Likewise, performing multiple assays with different complements of substrates (e.g., all different substrates, some different substrates, some overlapping substrates) can provide the same information (i.e., have the same breadth of substrate coverage) as a protein array. Furthermore, selection of sufficiently distinct characteristics for each substrate permits one to assay for numerous protein kinases or numerous phosphatases without costly equipment, for example simply by performing SDS-PAGE.

In general, the compositions of this aspect of the invention comprise one or more substances that are compatible with stable maintenance of the substrates of the invention for a sufficient amount of time for the substrates to be used in an assay. Examples of such substances include, but are not limited to, water, biologically compatible buffers and salt solutions, proteinase inhibitors, organic solvents, sugars, and the like, and mixtures of two or more of these. While it is preferable that the compositions comprise substances that can be present in the assay mixtures of the invention, it is not necessary that the substances be such. When the compositions comprise substances that are not compatible with the assay mixtures, such substances can be removed prior to use of the substrates of the invention in the assays. Removal can be accomplished by any known technique that retains the activity of the substrates.

Compositions comprising a substrate of the invention can, but do not necessarily, include a sample or biological cell that is suspected of containing a protein kinase or protein phosphatase of interest. That is, a composition comprising a substrate of the invention is not limited to purified substrates, but can include substrate in the presence of test materials, such as, but not limited to, intact cell material and cell lysate material (either crude lysate or partially purified lysate that contains a protein fraction).

Thus, a composition according to the invention can comprise at least two different protein kinase or protein phosphatase substrates, where the substrates independently comprise a tag and at least one target site specific for a protein kinase or a protein phosphatase, wherein all target sites present on one substrate are specific for the same protein kinase or protein phosphatase, each substrate is specific for a different protein kinase or set of protein kinases that share a common target site, or is specific for a different protein phosphatase or set of protein phosphatases that share a common target site, and each substrate differs in size from every other substrate. In preferred embodiments, the composition comprises at least two substrates that comprise a tag that is glutathione-S-transferase, biotin, or hexahistidine. In embodiments, each substrate independently comprises between 1 and 9 target sites. In embodiments, the target sites specific for each protein kinase or each protein phosphatase are different sizes. As mentioned above, in embodiments, the compositions comprise a solid support that comprises a binding partner for the tag, which allows purification of the substrates.

In a third aspect, nucleic acids are provided that encode a substrate of the invention. The nucleic acids can be any type of nucleic acid that is known to be suitable for expression of a protein product or maintenance of nucleic acid materials, and can include non-nucleic acid material (e.g., peptide nucleic acids or polyamide nucleic acids) or modified bases. Thus, the nucleic acid of the invention can be double stranded or single stranded, linear or circular, monomer or concatamer, DNA or RNA, pure nucleic acid or a nucleic acid/protein complex. Accordingly, it can be a self-replicating nucleic acid or a non-replicating nucleic acid that is present for only a short period of time when introduced into a cell. It thus may be a naked nucleic acid (such as a plasmid) or a packaged nucleic acid, such as one present in a viral particle or associated with viral proteins, or one present in a liposome or other carrier molecule. Those of skill in the art are aware of various forms of nucleic acids that are suitable for maintenance and/or delivery to cells, and all of the known forms are encompassed by the present invention. Because the nucleic acids of the invention may be suitable for expression in a cell, they can contain some or all of the elements necessary for maintenance and/or expression of the nucleic acid in a cell (prokaryotic or eukaryotic). Such elements are well known to those of skill in the art, and thus need not be detailed here. Selection of appropriate maintenance and expression elements can be performed by those of skill in the art without undue experimentation.

The nucleic acids generally comprise sequences that encode at least one target site for a protein kinase or protein phosphatase. As detailed above, substrates according to the present invention can comprise a single target site or target site unit, or can comprise two or more target sites or target site units. While it is possible to chemically synthesize each target site or target site unit as individual peptides, then fuse the peptides to each other and a tag to create a substrate according to the invention, and while it is possible to express multiple target sites or target site units and a tag from individual nucleic acids, then fuse the resulting peptides together to create a substrate according to the invention, it is preferred to design a single nucleic acid that encodes the correct number of target sites (with the appropriate target sequence) and a tag, in the same reading frame, and express the entire single nucleic acid to produce a single substrate as a fusion protein. Techniques for achieving substrates in any of these ways are well known to those of skill in the art and can be applied in the present invention without undue experimentation. Accordingly, such techniques need not be detailed here.

In embodiments, nucleic acid sequences encoding a known target site for a known protein kinase or a known protein phosphatase are designed such that the resulting expressed peptide has a known sequence and a known molecular weight. While it is possible to use any of a number of techniques to create such a target site, it is preferred to use the polymerase chain reaction (PCR) to amplify a region of nucleic acid encoding a known and previously characterized target site. Various modifications to the amino acid sequence surrounding the target site can be made during or after PCR amplification, the changes being made for any reason, such as to provide a convenient size for the encoded peptide, to insert or destroy other binding sites for other proteins (other than protein kinases or protein phosphatases), or to stabilize the secondary or tertiary structure of the peptides when the peptides are present alone or as part of concatamers and/or within the overall structure of the ultimate substrate. Those of skill in the art are well aware of techniques and considerations to achieve such modifications, and thus can make such modifications without undue experimentations and without the need for a detailed description here.

In general, in preferred embodiments, nucleic acids of the invention comprise a sequence encoding at least one target site fused in-frame to a sequence encoding a tag. This fusion construct is placed under the control of elements necessary for its expression in a prokaryotic cell or a eukaryotic cell. The nucleic acid preferably comprises elements necessary for maintenance of the nucleic acid in a host cell, such as elements necessary for replication of the nucleic acid or elements necessary for insertion of the nucleic acid into the genome of the host cell. In certain embodiments, multiple sequences encoding a single target site are provided, arranged in a liner fashion such that multiple repeats of the target site are expressed as linear repeats in the encoded primary amino acid sequence. As discussed above, the target site may be present within a larger coding region referred to as a target site unit. By providing constructs encoding either a single target site or multiple target sites, various substrates can be produced, each having a unique molecular weight.

The nucleic acids of the invention can be used to produce substrates for use in vitro or in vivo. When produced for use in vitro, the nucleic acids can be introduced into a host cell, for example E. coli, then expressed. The encoded substrates are then optionally purified away from some or all of the other cellular components before use. The nucleic acids can also be expressed in vitro, for example by using commercially available in vitro transcription and translation systems, then the encoded substrates optionally purified away from some or all of the components present in the in vitro system before use. Typically, when produced for use in vitro with at least one other substrate, a single substrate is encoded by a single nucleic acid of the invention, then, after production and optional purification, combined with the other substrates to create a composition.

When produced for use in vivo, the nucleic acids can be introduced into a host cell, for example a human cancer cell, then expressed. The nucleic acids can encode a single substrate or can encode multiple substrates as discrete, separate proteins. When a nucleic acid encodes a single substrate, in order to assay multiple protein kinases or phosphatases in a single cell, multiple nucleic acids, each encoding a substrate specific for a different protein kinase or protein phosphatase, should be introduced into the cell. Where the nucleic acids are to be stably maintained in the cell, introduction of each nucleic acid can be accomplished at the same time or sequentially, after confirmation of successful integration of one or more other nucleic acids. On the other hand, introduction of a single nucleic acid that encodes all of the desired substrates (in all desired concatamer forms) can be preferable. In this embodiment, each coding region for each substrate preferably is operably linked to control elements that are sufficient for expression of the coding region (including the tag, if appropriate). Preferably, the identity of each control element is the same for each coding region. In this way, multiple substrates can be expressed in essentially equal amounts, and there is no need to check or control for copy number effects, as would be the situation when substrates are introduced and expressed using multiple different nucleic acids and control elements. Compositions comprising the nucleic acids and substrates of the invention within a living cell provide an excellent way of identifying active protein kinases or protein phosphatases under in vivo conditions.

Thus, the invention provides a nucleic acid encoding at least two different protein kinase or protein phosphatase substrates in which the encoded substrates each comprise a tag and at least one target site specific for a protein kinase or a protein phosphatase, where all target sites present on one substrate are specific for the same protein kinase or the same protein phosphatase, where each substrate is specific for a different protein kinase or a different protein phosphatase, and where each substrate differs in size from every other substrate. In embodiments, expression of each encoded substrate of the nucleic acid of the invention is controlled independently of each other encoded substrate.

In a fourth aspect, the invention provides methods of making nucleic acids according to the invention. In general, the method comprises engineering a nucleic acid sequence encoding an amino acid sequence of interest (for convenience, this will be called a “fragment” from here on); synthesizing a double-stranded fragment; ligating the fragment in-frame to one or more other fragments, to a heterologous, relatively short nucleic acid (such as one encoding a tag or a stuffer), or to a vector capable of expressing the fragment(s) and optional other nucleic acid (such as one encoding a tag or stuffer); cleaving ligation products containing incorrect ligation products; and optionally purifying the correct ligated constructs from cleavage products. The resulting purified constructs can be used directly to express the substrates of the invention (e.g., by in vitro transcription/translation, by introduction into host cells for expression), or can be amplified before use for expression.

Engineering (or designing) the fragment includes identifying, from public databases or personal knowledge, amino acid sequences of interest, such as protein kinase or protein phosphatase target sites. Those of skill in the art are well versed in techniques for identifying sequences of interest. Thus, exemplary techniques, such as searching public sequence databases and using computer programs to design primers, need not be detailed here. Any suitable technique for identifying a sequence of interest and designing primers may be used.

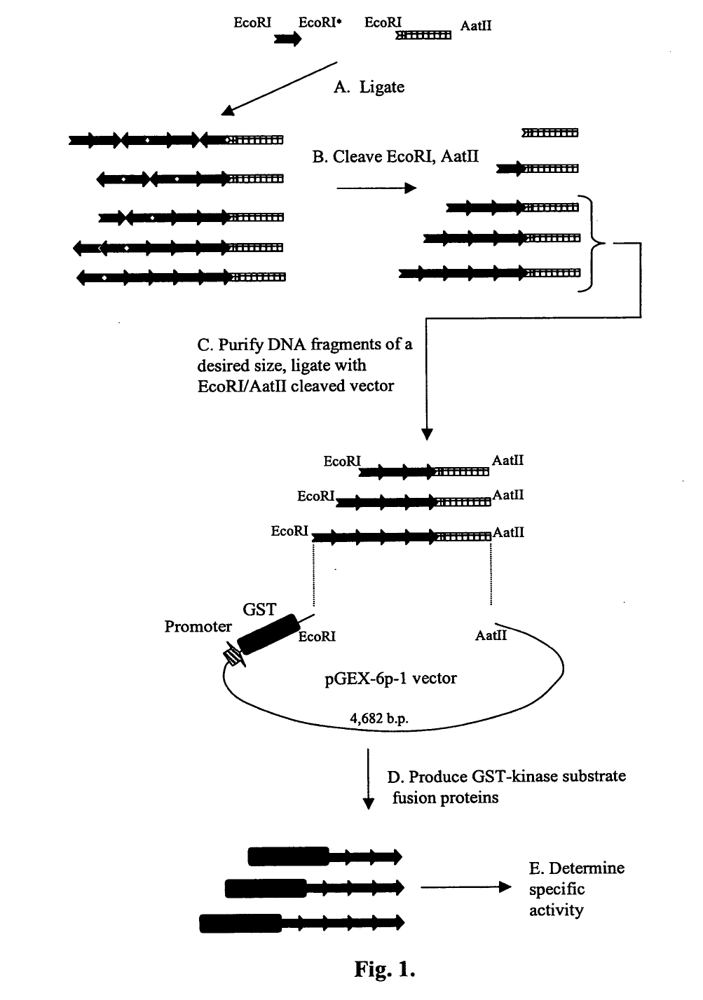

The fragment is designed such that a first end is a restriction endonuclease cleavage site product (i.e., the end comprises nucleotides on both strands that would remain after cleavage by an endonuclease) that can be ligated with another corresponding restriction endonuclease cleavage site product to form a new endonuclease cleavage site (i.e., a “competent” site capable of being correctly cleaved by an endonuclease). The other end of the fragment is engineered to have an “incompetent” restriction endonuclease cleavage site product (typically for the same endonuclease as designed on the first end). By “incompetent” site product, it is meant that the restriction site product, while resembling a restriction endonuclease cleavage site, does not form a restriction endonuclease cleavage site if ligated to a corresponding cleavage site product. For example, EcoRI recognizes the sequence 5′-GAATTC-3′, and cleaves this to release two nucleic acid ends, one having an ending sequence 5′-G (the G being base-paired to a C on the complementary strand) and the other (on the complementary strand) having an ending sequence of 5′-AATTC (the C being base-paired to a G on the complementary strand). In this example, a fragment of the present invention would include one end having the appropriate (competent) ending sequence of 5′-AATTC, and the other end having an inappropriate (incompetent) ending sequence of 5′-AATTG (other inappropriate sequences could be designed easily). While both ending sequences will base-pair with a (competent) EcoRI restriction endonuclease cleavage site product on a different nucleic acid, the incompetent ending sequence will cause the hybridized product, upon ligation to have a sequence (5′-CAATTC-3′) that is not a recognition sequence for EcoRI. Thus, ligation creates an “incompetent” or incomplete EcoRI site. (See FIG. 1.)

Creation of fragments that, when ligated to other nucleic acids, form one competent and one incompetent restriction endonuclease site permits directional cloning of the fragments into other nucleic acids. That is, because fragments cloned in one direction will lack a competent endonuclease cleavage site, they are resistant to cleavage by a particular endonuclease, and thus remain uncleaved upon exposure to the endonuclease, whereas fragments cloned in the opposite orientation (which comprise a competent endonuclease site) are cleaved when exposed to the endonuclease. Because the fragments can be directionally cloned, they can be engineered such that their coding sequences are in-frame with the coding sequences of other nucleic acids to which they are to be ligated. For example, if concatamers of target sites or target site units are desired, multiple fragments can be ligated together, resulting in in-frame fusion of the fragments, and the possibility to express repeating target sites or target site units.

The concept of creating fragments having one end that is a competent restriction endonuclease site product and one end that is an incompetent restriction endonuclease site product can be applied to any restriction endonuclease and for any set of fragments. It can be applied to endonucleases that leave a 5′ overhang, that leave a 3′ overhang, or that leave blunt ends. The length of the overhang is also not relevant to the present directional cloning concept. Likewise, the “incompetent” site can be created by either addition or deletion of one or more nucleotides to the endonuclease cleavage site product. The only practical limitation is that a sufficient number, in the proper sequence, of nucleotides should remain to permit association of the two nucleic acid ends to permit ligation. Of course, this limitation does not apply where blunt-end restriction endonuclease sites are involved. Thus, this technique can be used to produce numerous amino acid sequences for any number of purposes. Those of skill in the art will immediately recognize the wide applicability of this technique, and the many different uses for it when directional cloning of fragments is desired. Thus, although the present invention is exemplified with fragments encoding protein kinase or protein phosphatase target sites and target site units, the directional cloning methods of the invention are not limited to such uses. Thus, although the present disclosure might refer to cloning of a “fragment” of the invention into other nucleic acids, it should be understood that such a disclosure is for exemplary purposes only, and does not limit the scope of the applicability of this technique.

The fragment is engineered such that association and ligation of the incompetent site product to a competent site product creates an incompetent site that is resistant to later cleavage by the endonuclease. The fragment can also be designed such that the ligation product that creates an incompetent endonuclease site creates an in-frame fusion of the fragment to another nucleic acid, permitting not only directional cloning, but in-frame cloning as well. The fragment design also eliminates association and ligation of two incompetent endonuclease site products (e.g., two sequences ending in 5′-GTTAA will not hybridize, and thus will not be subject to ligation). In addition, the nucleic acid is designed such that association and ligation of two competent endonuclease sites (either from two engineered molecules or from an engineered molecule and a vector, etc.) forms a competent site. In the method, multiple copies of the engineered sequence are mixed with a sequence encoding a tag or a nucleic acid vector comprising a sequence encoding a tag, and the various nucleic acids are ligated together. The resulting constructs, the population of which comprises different numbers of engineered molecules inserted into the vector, is then digested with the restriction endonuclease for which the engineered nucleic acids have sites or incompetent sites. Cleavage of the competent sites occurs, leaving only constructs having one or more engineered sequences fused in-frame with each other and with the tag, all. If the tag and engineered molecules were ligated in the absence of a vector, the resulting in-frame construct can be digested with the appropriate nuclease(s) (if necessary) and ligated into the vector. In embodiments, the engineered sequences encode target sites or target site units.

The method further includes synthesizing a nucleic acid of interest. Synthesizing can be by any of the numerous techniques available, any of which can be used without undue experimentation. One preferred technique is copying amplifying a known sequence or a sequence having portions that are known (such that primers can be designed that are specific for the sequence). While any technique for amplifying the nucleic acid sequences can be used, typically, PCR is used in conjunction with primers that are specific for the nucleic acid encoding the amino acid sequence of interest, and that contain sequences that will form restriction endonuclease site products and “incompetent” (or incomplete) restriction endonuclease site products, as detailed below. Numerous PCR techniques are available, and any such techniques can be used without undue experimentation. Synthesizing preferable results in a double-stranded product.

As evident from the disclosure above, the method comprises ligating the fragment into one or more other nucleic acids. Ligation of an incompetent restriction endonuclease site product to a competent site product produces an incompetent site that is resistant to cleavage by the endonuclease for which the competent site product is specific. Ligation of two competent site products creates a competent site that is susceptible to cleavage by the endonuclease. Ligation of two incompetent site products is precluded by the design of the site products. Ligation can be performed using any known ligase and under any suitable condition. A preferable ligase is T4 ligase, available with instructions for use from numerous commercial suppliers.

Ligation can include ligating one or more fragments to other nucleic acids. The other nucleic acids can be other nucleic acids of interest, such as those encoding a tag or stuffer. The other nucleic acid can also be a vector, such as a vector suitable for expression of peptides in cell. Numerous such vectors are commercially available, and any known vector can be used. Typically, such vectors include all of the elements necessary to express a peptide in a cell of choice, and include both transcription and translation control and initiation sites. In certain vectors, the codon for the initial N-terminal amino acid(s) is included to facilitate expression and to provide sites for in-frame cloning. In embodiments, the vector comprises a sequence encoding a tag according to the invention, which can be expressed from the vector from appropriate expression elements on the vector. Ligation of the fragment(s) to the vector occurs at the tag, resulting in an in-frame fusion of the tag and fragment(s), the tag being located either at the 5′-end of the fusion construct (the N-terminus of the encoded fusion protein) or 3′-end (C-terminus of the encoded fusion protein). Ligation can also include ligation of two or more fragments in tandem (and in-frame). Ligation of each individual nucleic acid (fragment(s), tag, vector) can be in any order or can be accomplished simultaneously. Some or all of the ligations may be performed separately, together, or in various combinations. Thus, although it is preferable to have the sequences encoding the tag at either end of the fusion product, it is possible to insert the tag between two (or more) fragments.

Optionally, the fragments are purified (or cleaned) after the ligation reaction. Numerous methods and compositions for purifying nucleic acids are known and/or commercially available, and any suitable method and compositions can be used.

After ligation, the products are subjected to cleavage by at least the restriction endonuclease for which the “competent” sites are specific. Depending on the needs of the practitioner, other endonucleases can be used to cleave within the fragment or other nucleic acids. Such multiple cleavages can produce convenient cloning sites, can be used to identify sizes of inserts, or to provide any other useful information about the constructs and the synthesis and ligation processes. Cleavage of the products results in cleavage of all competent endonuclease sites. Ligation products having incompetent sites (i.e., those having the proper directional cloning) are resistant to cleavage at the ligated sites. Thus, cleavage results in constructs having the desired directional cloning, and eliminates constructs that have undesirable cloning products (i.e., those having fragments in the wrong orientation).

In embodiments where the products are to be expressed in a vector, ligation may be performed with the vector present with the fragment or various fragments and/or other nucleic acids to be ligated. Alternatively, the fragment(s) and optional other nucleic acids can be ligated, cleaved, purified, and ligated into the vector, which has been digested with the appropriate restriction endonuclease(s) for cloning of the products. The products may be cloned into the vector at the appropriate sites using any suitable technique known to those of skill in the art. The vectors will have the characteristics discussed above.

The desirable ligation/cleavage products (including those comprising a vector) can optionally be purified (or cleaned) after cleavage and/or after re-ligation. Numerous methods and compositions for purifying nucleic acids are known and/or commercially available, and any suitable method and compositions can be used. Exemplary techniques include, but are not limited to, density gradient centrifugation, such as through a salt solution (e.g., cesium chloride (CsCl)) or a sugar solution (e.g., sucrose); separation through a gel matrix based, at least in part, on size (e.g., gel electrophoresis), differential precipitation, and various commercially available products for purifying nucleic acids.

The resulting ligation/cleavage products have any number of uses, including any use that one of skill in the art would understand to be applicable for nucleic acids. According to the exemplary embodiments of the invention, the products are vectors comprising sequences encoding an expressible tag fused in-frame to at least one target site for a protein kinase or protein phosphatase.

In an exemplary embodiment, a double-stranded DNA oligonucleotide-encoding polypeptide substrate is synthesized, concatamerized, cloned in-frame with an ATG codon, and cloned into a DNA vector. Such a procedure allows synthesis of a polypeptide containing several enzyme target sites in an appropriate host cells. In this embodiment, the polypeptide is expressed in fusion with a protein tag, which facilitates the polypeptide-tag fusion protein purification, detection, and immobilization on a solid support. Multiple such polypeptides of differing sizes, each containing different enzyme recognition amino acid sequences, can be combined in a mixture to assess enzyme activity in a multiplex assay. After enzymes react with the substrate mixture and the substrates are purified away from the rest of the components in the reaction mixture, the various components of the mixture can be separated by size.

As discussed above, when multiple different substrates are to be provided in a single composition, it is preferable that each substrate have a unique molecular weight to facilitate identification of the substrate, and thus the protein kinase or protein phosphatase acting upon the substrate. Using standard molecular biology techniques, it is a straightforward matter to design and create such unique substrates. For example, one may design a target site unit that is specific for protein kinase C (PKC) that contains 13 amino acid residues (which results in a peptide having a molecular weight of approximately 1,300 daltons (Da). A substrate containing two such units will have a molecular weight of approximately 2,600 Da; a substrate containing three such units will have a molecular weight of approximately 3,900 Da; and so forth. Concurrently, one may also design a target site unit that is specific for protein kinase A (PKA) that contains 20 amino acid residues (which results in a peptide having a molecular weight of approximately 2,000 Da). A substrate containing two such units will have a molecular weight of approximately 4,000 Da; a substrate containing three such units will have a molecular weight of approximately 6,000 Da; and so forth. Such a strategy can be followed for any number of protein kinases, resulting in a set of substrates (and substrate ladders) that have unique molecular weights, and thus can be easily detected and identified, and correlated with a particular protein kinase. Of course, if a tag is to be included in the substrate and not cleaved before detecting the presence of labeled substrate, the molecular weights would be increased by an amount corresponding to the molecular weight of the tag. Likewise, substrates and substrate ladders can be created for particular protein phosphatases in the same manner as they are created for protein phosphatases. However, an additional step of phosphorylating the substrates will be required to provide a suitable substrate for phosphatases.

As is evident from the above discussion, the invention also provides compositions comprising nucleic acids of the invention. The compositions can include multiple copies of the same nucleic acid. Alternatively, the compositions can comprise multiple nucleic acids, each encoding the same target site or target site unit, but a different number of times. Furthermore, the compositions can comprise multiple different nucleic acids, each encoding a different target or target site specific for a different protein kinase (or group of protein kinases having the same target site specificity) or a different protein phosphatase (or group of protein phosphatases having the same target site specificity). In embodiments, the compositions comprise a combination of two or more different nucleic acids, having different numbers of target sites or target site units, encoding target sites that are specific for different protein kinases or different protein phosphatases, and encoding different tags. Any combination of target site specificity, target site number, and tag are encompassed by the compositions of the present invention.

In general, the compositions comprising a nucleic acid comprise one or more substances that are compatible with stable maintenance of the nucleic acids of the invention for a sufficient amount of time for the nucleic acids to be used. Such substances are well known to those of skill in the art, and thus need not be detailed here. The compositions typically, but not necessarily, include an aqueous buffer compatible with other components used to insert nucleic acids into cells, substances present in in vitro transcription assay mixtures, substances present in acellular amplification (e.g., PCR) mixtures, or cellular components present in cells or in cell lysates. The compositions can also comprise substances that promote uptake of the nucleic acids into host cells, such as viral particles, viral proteins, liposomes, and the like.

Thus, the present invention provides a composition comprising at least two different nucleic acids encoding protein kinase substrates or protein phosphatase substrates, where the nucleic acids independently encode different substrates comprising a tag and at least one target site specific for a protein kinase or a protein phosphatase, where all target sites present on one substrate are specific for the same protein kinase or the same protein phosphatase, where each substrate is specific for a different protein kinase or a different protein phosphatase, and where each substrate differs in size from every other substrate. In embodiments, the composition comprises at least one cell lysate.

In another aspect, the invention provides cells comprising a substrate of the invention, a nucleic acid of the invention, or both. While the cells can be considered as components of certain compositions of the invention, they may also be considered as a separate aspect of the invention. Cells of the invention are referred to herein interchangeably simply as cells or as host cells. The cells of the invention are generally useful for expressing the substrates of the invention for either purification and later use in in vitro or for in vivo assays. They are also generally useful for producing more (i.e., amplifying) a nucleic acid of the invention and for maintaining a nucleic acid of the invention. The cells of the invention can comprise a nucleic acid of the invention as an autonomously replicating entity (e.g., a plasmid) or as an integrated part of the cell's genome. A cell according to the invention can be either prokaryotic or eukaryotic. Numerous prokaryotic and eukaryotic cells that are suitable for introduction and maintenance of heterologous nucleic acids are known in the art, and all such cells are encompassed by this invention. Likewise, numerous techniques for introducing exogenous and heterologous nucleic acids into prokaryotic and eukaryotic cells are known in the art, and all such techniques are encompassed by this invention.

In particular embodiments, the cells of the invention are used for in vivo assays for protein kinases or protein phosphatases. In these embodiments, one or more nucleic acids of the invention are introduced into the cells, substrates encoded by the nucleic acids are expressed, and the presence of one or more protein kinases or protein phosphatases present in the cell is detected through detection of the activities of the protein kinase(s) or protein phosphatase(s). In this way, one or more protein kinases or one or more protein phosphatases can be detected in the cell. In embodiments, the cell is a eukaryotic cell, such as one associated with a disease or disorder, such as a cancer cell, an immune system cell, or a cell infected with a virus, bacterium, or intracellular parasite. In other embodiments, the cell is a prokaryotic cell, such as one associated with a disease or disorder. In embodiments, the cell is a normal, healthy prokaryotic or eukaryotic cell.

In yet another aspect of the invention, assays are provided. In one embodiment, the assays provide information on the presence of one or more protein kinases in a sample, such as in a cell or a cell lysate. The assays of this embodiment rely on detection of protein kinase substrates, and in particular, protein kinase substrates that have been modified, such as by phosphorylation, through the specific action of one or more protein kinases. In another embodiment, the assays provide information on the presence of one or more protein phosphatases in a sample, such as in a cell or a cell lysate. The assays of this embodiment rely on a loss of detection of protein phosphatase substrates, and in particular, protein phosphatase substrates comprising labeled phosphate groups.

The assays generally comprise providing at least one protein kinase substrate or at least one protein phosphatase substrate having a known target site; providing a sample containing or suspected of containing at least one protein kinase for which a target site has been defined, or at least one protein phosphatase for which a target site has been defined; combining the substrate and the sample for a sufficient amount of time for modification of the target site to occur if a suitable protein kinase or protein phosphatase is present, and determining if a modified substrate is present. In the case of a kinase assay, the presence of a phosphorylated substrate is indicative of the presence of a specific protein kinase in the sample being tested. In the case of a phosphatase assay, the loss (partial or total) of signal due to a phosphate-labeled substrate is indicative of the presence of a specific protein phosphatase in the sample being tested.

Providing at least one substrate can be accomplished in many ways, as detailed above. In general, providing comprises making the substrate and supplying it in a form that is satisfactory for use in an assay of the invention (e.g., in an appropriate concentration and in conjunction with other substances that are compatible with the assay conditions). A preferred way of providing the substrate is to provide a nucleic acid encoding the substrate, and expressing the nucleic acid to produce the substrate. Expression can be performed in vivo, such as in a transformed host cell, or in vitro, such as by acellular amplification (e.g., PCR) followed by in vitro transcription and translation.

In embodiments, particularly embodiments where the assay will be performed entirely in vitro, the expressed substrate can be purified, either partially or completely, from the other substances that are present in the composition used to produce the substrate. Purification can be by any known technique, including those typically used to purify proteins (e.g., column chromatography, size exclusion, filtration, precipitation, affinity purification, and the like). It is often convenient to use affinity purification, based on binding of the tag portion of the substrate, to purify the substrate after making it. After purification, the affinity purified substrate may be released from the affinity purification support (e.g., solid support resin, bead, etc.) or used directly in its solid support-bound form.

In embodiments where the assay is performed at least in part in vivo, providing the substrate typically comprises introducing at least one nucleic acid of the invention into a host cell, and expressing the substrate in the host cell. As discussed above, the substrate(s) can be provided as expression products from a single nucleic acid construct (e.g., plasmid) or from multiple nucleic acid constructs, and the various nucleic acid constructs can be autonomously replicating particles or integrated into the host cell's genome. In a preferred embodiment, multiple nucleic acid constructs are introduced into a host cell and expressed to produce multiple different substrates. The amounts of each substrate (modified or not) present in the assay are normalized during the detecting portion of the method (e.g., by normalizing gel intensity of the substrate bands based on characteristics not related to the labeling, such as by protein staining).

As described above, numerous substrates can be used in the assays of the invention. The substrates can be all of one kind, having the same primary amino acid sequence and being specific for the same protein kinase or protein phosphatase. Alternatively, the substrates can all be specific for one protein kinase or one protein phosphatase, but have different numbers of target sites or target site units. Alternatively, the substrates can comprise two or more different substrates that are specific for two or more different protein kinases or two or more different protein phosphatases, each substrate independently having a pre-selected number of target sites or target site units and provided in a pre-selected concentration or amount. Numerous permutations of individual substrates can be provided, and all such permutations are encompassed by this invention.

The substrates can be provided in any amount and in any form. However, it has been found that in vitro assays perform well when each substrate is provided as a purified (at least to some extent) product, and is present in the assay mixture in an amount of about 0.1 μg to about 3.0 μg, such as about 0.1 μg, about 0.2 μg, about 0.25 μg, about 0.3 μg, about 0.4 μg, about 0.5 μg, about 0.6 μg, about 1.0 μg, about 1.5 μg, about 2.0 μg, and about 2.5 μg. Amounts less than about 0.1 μg are also contemplated by this invention, as are amounts more than about 3.0 μg. When multiple substrates are provided, it is preferable that they each be purified (at least to some extent) and provided in an amount from about 0.1 μg to about 0.75 μg.

According to the assays of the invention, providing a sample containing or suspected of containing a protein kinase or containing or suspected of containing a protein phosphatase can be accomplished in many ways. Typically, a sample containing biological material is obtained and, preferably, maintained in a state under which protein degradation is blocked or minimized (e.g., by addition of proteinase inhibitors, by maintaining the sample at a low temperature, etc.). The sample can be one that is known to contain a protein kinase or a protein phosphatase of interest, or can be one that is suspected of containing a protein kinase or protein phosphatase of interest. In embodiments where a negative control is to be performed, the sample can be one that is known not to contain a protein kinase or protein phosphatase that will specifically react with one or more substrates provided in the assay. Optionally, the sample may be subjected to one or more purification steps to purify, either partially or completely, one or more protein kinases or one or more protein phosphatases, or to obtain a protein kinase-containing or protein phosphatase-containing fraction. Likewise, proteins, protein kinases, or protein phosphatases can optionally be concentrated in the sample. Purification and/or concentration can improve the ultimate signal generation of the assay and can shorten the time required to generate a detectable signal.

The sample itself can be any of a number of things. Exemplary samples include, but are not limited to, cells (prokaryotic or eukaryotic), cell lysates or extracts of cell lysates (prokaryotic or eukaryotic, in any stage of purification), body fluids, tissue homogenates (or extracts in any stage of purification), and samples comprising one or more purified or partially purified components (typically to be used as positive or negative controls in the assays). In accordance with the discussion above, the term sample includes the interior of a living cell. Thus, the methods encompass in vivo as well as in vitro assays, and include assays that are performed partially in vivo and partially in vitro.

Combining the sample and the substrate permits the substrate to come in contact with any protein kinase or protein phosphatase present in the sample. According to the invention, the sample and substrate are maintained in a combination under conditions and for a sufficient amount of time for a detectable change in the substrate to occur (if a kinase or phosphatase that is specific for the substrate target site is present). Suitable conditions and times for protein kinase and protein phosphatase reactions to occur are numerous and widely known in the art. Any suitable conditions and times may be used in the assays of the present invention, and selection of suitable conditions and times is well within the level of skill of one of skill in the art. Thus, a detailed listing of the various conditions and times need not be presented here. Exemplary conditions are presented in the examples that follow.

Combining may be effected by any suitable technique, including, but not limited to, mixing by pipetting, stirring, inversion, or vortexing. It is to be noted that combining creates a composition according to the invention. In embodiments where substrate is bound to a solid support, it is often advantageous to provide continuous or periodic mixing of the composition to facilitate interaction of protein kinase(s) or protein phosphatase(s) with the substrate.

Determining if a substrate has been modified is effected by detecting a change in one or more substrates present in the assay mixture. A detectable change in the substrate is typically a modification of the substrate. Any modification that is detectable is envisioned by the present invention, including modifications made by protein kinases or phosphatases having activities in addition to protein phosphorylation or dephosphorylation, respectively (including engineered protein kinases having additional enzymatic activities engineered into them). However, due to the intrinsic, natural activity of protein kinases, it is preferred that the modification in a protein kinase assay be protein phosphorylation. Likewise, due to the intrinsic, natural activity of protein phosphatases, it is preferred that the modification in a protein phosphatase assay be protein dephosphorylation.

Detection of a change can be through any known technique, but is typically through assaying for the presence or absence of a phosphate group covalently bound to the target site. Numerous assays for phosphoproteins are known in the art, and any of these can be used in the present invention. Exemplary assays include, but are not limited to, detection of labeled phosphate groups (e.g., by radioactive emission, luminescence, phosphorescence, or fluorescence), detection of phospho-amino acids with anti-phosphopeptide antibodies, detection of phosphopeptide amino acids by total amino acid analysis, and the like. Other exemplary techniques include, but are not limited to, 2-dimensional gel electrophoresis, size exclusion chromatography (gel filtration), size exclusion filtration (e.g., through membranes), centrifugation (alone or in conjunction with size exclusion techniques), partial or total proteolysis to identify phosphopeptides, and mass spectroscopy. It is preferred that detection be performed through the use of common techniques, such as those that rely on PAGE.

In certain embodiments, the substrate(s) is purified, either partially or completely, before providing it, combining it with the sample, or determining if a substrate has been modified. Purification can be effected by any suitable technique, including, but not limited to, by affinity binding of the tag to its binding partner (which is bound to a solid support); affinity purifying by passing the substrate over a column comprising an affinity binding group. In certain embodiments, the substrate(s) is purified by binding of the tag to a binding partner associated with a solid support, such as through binding of a GST tag to a glutathione-containing solid support.

In certain embodiments, the substrate(s), sample, or combination is washed to remove unbound label, non-specifically bound proteins or other macromolecules, and other substances that could interfere with performance of one or more steps of the assay. Washing solutions and protocols for enzyme activity assays and affinity purification procedures are well known to those of skill in the art, and thus need not be detailed here. Any suitable washing protocol may be used in accordance with the present invention. An exemplary washing protocol is described in the examples below.

As discussed above, in certain embodiments, the assay of the invention is performed with the substrate bound to a solid support through binding of the tag to a binding partner associated with the solid support. Numerous binding pairs that are suitable for use as a tag and solid support-bound partner are known to those of skill in the art, and any of the known pairs can be used in the present invention. An exemplary pair (GST and glutathione) are detailed in the examples below.

Numerous protein kinases and protein phosphatases can be assayed using the methods of the present invention. There are currently well over one hundred known protein kinases that are known to be involved in specific cellular functions. The target sites for many of these have been characterized. Doubtless, many more will be characterized in the future. Likewise, numerous protein phosphatases and corresponding target sites have been identified. Due to its versatility, the present invention permits the user to design any number of substrate/kinase or substrate/phosphatase combinations for use in the invention. Indeed, as long as a protein kinase or protein phosphatase has been identified, and its target sequence identified, it can be assayed using the methods of the present invention.

Exemplary protein kinases having known target sites include, but are not limited to, Csk homologous kinase or cell cycle checkpoint kinase 1 (CHK), casein kinase 1 (CK1), casein kinase 2 (CK2), epidermal growth factor receptor/kinase (EGFR), extracellular signal-related kinase (ERK), inhibitor of kappa B factor kinase (IKK), cJun N-terminal protein kinase 1 (JNK), mitogen activated protein kinase (MAPK), mitogen activated protein kinase 10 or Jun N-terminal protein kinase 3 (JNK3), protein kinase A (PKA), AMP-activated protein kinase (AMPK), dsDNA protein kinase (DNA-PK; p350), elongation factor 2 kinase (EF2K), focal adhesion kinase (FAK), G protein-dependent receptor kinase 1 (GRK), protein kinase C (PKC), cyclin dependent kinase 1 (CDK1; cdc2), ataxia-telangiectasia mutated gene (ATM), G protein-dependent receptor kinase and insulin receptor (GRK+insulin rec), Abl, and Src.

Of course, the assays of the invention contemplate use of positive and negative controls to ensure that the results obtained are correct. The positive and negative controls are performed substantially the same way as the assays for suspected kinases or phosphatases: exemplary negative controls can be run with the sample, but no substrate, or with substrate, but no sample; exemplary positive controls include a sample known to contain a protein kinase or protein phosphatase. Other control reactions may be performed as well to confirm that any or all substances present in the reaction or detection steps are suitable for use in the assay and functioning as expected.

The assays of the present invention can identify the activity of a specific protein kinase or a protein kinase that is a member of a group of kinases that share a common target site. In addition, the assays of the present invention can identify the activity of a specific protein phosphatase or a protein phosphatase that is a member of a group of phosphatases that share a common target site. Identification is generally considered a qualitative result; thus, the present invention provides qualitative assays for protein kinase and protein phosphatase activities. The assays also can provide information on the relative level of activity of one or more protein kinases or one or more protein phosphatases in a sample. Thus, the assays can identify a single protein kinase or protein phosphatase activity or can identify multiple, different protein kinase or protein phosphatase activities (i.e., a multiplex assay). Such information can be very valuable in determining suitable targets for drugs that can affect various cellular activities.

Thus, the present invention provides a method of detecting the presence of a protein kinase or a protein phosphatase. The method generally comprises providing at least two different protein kinase or protein phosphatase substrates, where the substrates independently comprising a tag and at least one target site specific for the protein kinase or the protein phosphatase, where all target sites present on one substrate are specific for the same protein kinase or the same protein phosphatase, and where each substrate differs in size from every other substrate or comprises a unique tag that differs from the tags present in each other substrate that is specific for a different kinase of phosphatase; providing a sample that contains or is suspected of containing a protein kinase or a protein phosphatase; combining the substrate and sample for an amount of time sufficient for a protein kinase or protein kinase, if present, to modify a substrate; and determining if a substrate has been modified. In embodiments, the method is performed in vitro, in vivo, or partially in vivo and partially in vitro. In embodiments, each substrate is provided in an amount of about 0.1 μg to about 3.0 μg, such as about 0.1 μg, about 0.2 μg, about 0.25 μg, about 0.3 μg, about 0.4 μg, about 0.5 μg, about 0.6 μg, about 1.0 μg, about 1.5 μg, about 2.0 μg, and about 2.5 μg. Amounts less than about 0.1 μg are also contemplated by this invention, as are amounts more than about 3.0 μg. When multiple substrates are provided, it is preferable that they each be purified (at least to some extent) and provided in an amount from about 0.1 μg to about 0.75 μg.

In some embodiments, one or more substrate is bound to a solid support either during the mixing step or some other time before detecting whether a modification of the substrate occurred. In preferred embodiments, determining if a substrate has been modified comprises performing SDS-PAGE of at least a portion of the combination of substrate and sample.

Providing proteins on solid supports, such as beads, has advantages for many applications. However, pipetting a bead slurry is typically less accurate than pipetting solutions, as a portion of the beads might settle to the bottom of the tube, clog the pipette tip, or stick to the tube walls. It has been found that advantageous results have been obtained when splashing of the beads on the walls or top of the tube is avoided. However, if this happens, pulse-spinning of the tube in a microfuge to bring the beads back into the buffer can avoid errors due to loss. Furthermore, it has been found that it is beneficial to take care to ensure that the slurry is evenly suspended throughout the buffer, as sedimentation of the beads has been observed in as little as 1 to 2 minutes. Immediately before each aliquot is removed from the kinase substrate tube, it is beneficial to gently pipet the slurry up and down several times. In addition, tips with a relatively large opening (e.g. #PT0230-96-TRYS tips from Coast Scientific, Inc.) have been found to provide adequate mixing capabilities.

Experiments have shown that, although any suitable amount of beads can be used, it is often advantageous to use at least 2 μl of substrate beads and perform reactions in very clear 500 μl tubes. It has been found that the use of clear tubes enables good visualization while pipetting and of bead pellets during wash steps.

Washing bead-bound substrates prior to running them on a gel can reduce the background from endogenous phosphorylation in cell lysates, which when used as the source of kinase activity might contain numerous labeled proteins in addition to the substrates of the invention. Nearly all of this background can be removed by washing the substrates. However, some kinases might bind to their phosphorylated substrates and thus might pellet out with the substrate beads. In these cases, a kinase auto-phosphorylation band may be visible (e.g., Src, which binds its phosphorylated substrate through an SH2 domain, will often co-purify with the substrate). These bands can easily be detected and differentiated from bands corresponding to the substrates of the invention.

It has been found that, if commercial kinases are added to the substrates in the absence of a complex protein mixture (e.g., cell lysate), then the washing step can typically be omitted. Most commercial kinases will auto-phosphorylate to some degree, which can be seen when phosphoproteins are detected from gels if the kinase is not first washed away. If the autophosphorylation bands are problematic (e.g., they correspond in size with the kinase substrate) then washing the kinase substrate reactions is recommended. It is noted that some commercially available kinases are provided as GST fusion proteins and these will be expected to have some binding to the substrate-carrying beads. In those cases, it might not be possible to wash away the kinase completely, and one might wish to choose a different tag or a substrate size that differs from the kinase fusion protein of interest.

In a final aspect, the present invention provides kits. Kits of the invention can comprise one or more substrates according to the invention, one or more nucleic acids of the invention, one or more compositions of the invention, or combinations of two or all of these. The kits generally are designed to provide some or all of the components needed to perform an assay according to the invention. Thus, the kits can, but do not necessarily, contain all of the components needed to perform an assay according to the invention. Generally, each substrate is contained in its own container; however, in embodiments, multiple (2, 3, 4, 5, 6, or more) substrates are contained in a single container. Furthermore, in general, each nucleic acid is contained in its own container; however, in certain embodiments, multiple nucleic acids are contained in a single container. Typically, the kits comprise a package, such as a box or the like that contains one or more containers holding components useful for practicing the assays of the invention. Furthermore, the kits typically contain written instructions, either as a separate document or as a label on the kit or on one or more containers.

In embodiments, the kits comprise one or more of the following substrates (labels are based on the kinases that specifically bind to target sequences in the substrate), each in its own container or two or more in a single container: CHK, CK1, CK2, EGFR, ERK, IKK, JNK, MAPK, JNK3, PKA, AMPK, DNA-PK, EF2K, FAK, GRK, PKC, CDK1, ATM, GRK+insulin rec, Abl, and Src. In accordance with the discussion above, each substrate can comprise one or multiple target sites, creating different size substrates specific for the same protein kinase. Likewise, each substrate can comprise a unique tag. Alternatively, each substrate that is specific for a given protein kinase can have the same tag (e.g., all substrates that contain target sites for PKA can comprise a GST tag, while all substrates that contain target sites for EGFR comprise a biotin tag, etc.). Thus, in embodiments, a kit of the invention comprises a single container containing EGFR, IKK, JNK3, and Src.

The various substrates can independently be provided in different amounts in the kit. Although any amount of each substrate can be provide in a kit, exemplary embodiments provide about 1 μl to about 1,000 μl, such as about 2 μl to about 100 μl, about 5 μl to about 50 μl, or about 10 μl to about 20 μl. The concentrations of substrates can likewise be individually varied (both with regard to substrates that have different kinase or phosphatase specificities and with regard to substrates that have the same kinase or phosphatase specificity, but differ in size or tag). Although any concentration of each substrate can be provided in a kit, exemplary embodiments provide each substrate in a container in a concentration of about 0.1 μg/μl to about 1.0 μg/μl, such as from about 0.125 μg/μl to about 0.625 μg/μl, about 0.250 μg/μl to about 0.5 μg/μl, or about 0.1 μg to about 0.2 μg/μl. In exemplary embodiments, mixtures of two or more substrates comprise about 0.125 μg/μl to about 0.250 μg/μl of each substrate in the container. In exemplary embodiments, a single substrate is contained in a single container at a concentration of about 0.5 μg/μl. When the substrates are provided as solid-phase bound compositions, such as GST fusion proteins bound to glutathione-agarose beads, they can be provided in any suitable formulation, such as, but not limited to, a 15% to 50% slurry in 40 mM Tris-HCl (pH 8.0), 120 mM NaCl, and 20% glycerol. The concentration of the substrate in such embodiments is calculated using the combined volume of beads and buffer.

Thus, the kits can comprise substrates that are specific for one or more protein phosphatases, each in its own container or two or more in a single container. As with the substrates for protein kinases, each substrate can comprise one or multiple target sites, creating different size substrates specific for the same protein phosphatase. Likewise, each substrate can comprise a unique tag. Alternatively, each substrate that is specific for a given protein phosphatase can have the same tag. Concentrations and amounts of substrates in the containers will generally be the same as for the protein kinase substrates. The protein phosphatase substrates can be provided as phosphorylated molecules or can be provided as non-phosphorylated molecules, which are to be phosphorylated by the practitioner prior to use. In embodiments where non-phosphorylated substrates are provided, the kits may contain some or all of the reagents and supplies necessary for phosphorylation and purification of the substrates.

The kits of the invention can comprise solid supports, such as beads, for use in binding the tag of the substrates. The solid supports can be provided in the same container as the substrate(s) or as a separate component in a separate container.

The kits of the invention can comprise one or more buffers or other compositions that can be used in the assays of the invention. The buffers can be stock compositions that are suitable for most, if not all, protein kinase and/or protein phosphatase assays, or can be optimized for a particular protein kinase or protein phosphatase and substrate combination. Thus, the kits can contain a reaction buffer for kinase and/or phosphatase reactions. A non-limiting exemplary reaction buffer comprises: 8 mM MOPS, pH 7.0 and 0.2 mM EDTA. A non-limiting solution for supplying ATP for phosphorylation reactions comprises: 20 mM MOPS, pH 7.2, 25 mM β-glycerol phosphate, 5 mM EGTA, 1 mM sodium orthovanadate, 1 mM dithiothreitol, 75 mM MgCl₂, and 500 μM ATP. Because buffers and solutions are often added as a component of a larger volume mixture, the buffers and other solutions can be supplied as concentrated compositions, such as 2×, 5×, 10×, 20×, or 50× solutions, to be diluted as needed before using or to be diluted to a final 1× concentration by the other components of the mixture.

The kits of the invention can also comprise a washing solution for washing away unbound label or substances present in the assay mixture that are not necessary for detection of modified substrates.

Other items that might be useful in practicing the invention may also be included in the kits of the invention.

### EXAMPLES

The invention will be further clarified by the following examples, which are intended to be purely exemplary of the invention, and should not be considered as limiting the invention in any way.

### Example 1

### Plasmid Constructs Encoding Protein Kinase Substrates

A protocol was developed for construction of plasmid vectors expressing protein kinase substrates (FIG. 1). pGEX-6p-1 vector (Amersham Pharmacia Biotech) was used as the parent vector to express protein kinase substrate peptides as carboxy-terminal fusions with GST. Synthetic oligonucleotides encoding protein kinase substrate peptides were designed based on the literature data (Table 1).

TABLE 1

Protein kinase peptide substrate and dsDNA oligonucleotide
sequences used to generate GST fusion kinase substrate proteins.*

| | |
|---|---|
| Kinase | Peptide and oligonucleotide sequences |

| | | | |
|---|---|---|---|
| 1 | Abl | A I Y A A P P | (SEQ ID NO: 7) |
| | | AATTCGCGATTTATGCGGCGCCGTTTC | (SEQ ID NO: 8) |
| | | GCGCTAAATACGCCGCGGCAAAGTTAA | (SEQ ID NO: 9) |
| 2 | CHK | D Q E A K V S R S G L Y R S P S M P E N L N | (SEQ ID NO: 10) |
| | | AATTCGATCAGGAAGCGAAAGTGAGTGCGAGTGGTCTGTATCGTTCTCCGTCTATGCCGGAAAACCTGAACC | (SEQ ID NO: 11) |
| | | GCTAGTCCTTCGCTTTCACTCACGCTCACCAGACATAGCAAGAGGCAGATACGGCCTTTTGGACTTGGTTAA | (SEQ ID NO: 12) |
| 3 | CK1 | D L H D D E E D E A M S I T A | (SEQ ID NO: 13) |
| | | AATTCGATCTGCATGATGATGAAGAAGACGAGGCGATGTCTATCACCGCAC | (SEQ ID NO: 14) |
| | | GCTAGACGTACTACTACTTCTTCTGCTCCGCTACAGATAGTGGCGTGTTAA | (SEQ ID NO: 15) |
| 4 | CK2 | R R D D D S D D D | (SEQ ID NO: 16) |
| | | AATTCCGTCGCGATGACGATAGCGATGATGACC | (SEQ ID NO: 17) |
| | | GGCAGCGCTACTGCTATCGCTACTACTGGTTAA | (SEQ ID NO: 18) |
| 5 | dsDNA | G G G E E T Q T Q D Q P M E E E E V | (SEQ ID NO: 19) |
| | kinase | AATTCGGCGGTGGCGAAGAAACCCAGACCCAGGATCAGCCGATGGAAGAGGAAGAAGTTC | (SEQ ID NO: 20) |
| | (DNA- | GCCGCCACCGCTTCTTTGGGTCTGGGTCCTAGTCGGCTACCTTCTCCTTCTTCAAGTTAA | (SEQ ID NO: 21) |
| | PK | | |
| 6 | EGFR | A E N A E Y L R V A P | (SEQ ID NO: 22) |
| | | AATTCGCAGAAAACGCAGAATATCTGCGTGTGGCACCGC | (SEQ ID NO: 23) |
| | | GCGTCTTTTGCGTCTTATAGACGCACACCGTGGCGTTAA | (SEQ ID NO: 24) |
| 7 | ERK | K Q A E A V T S P R | (SEQ ID NO: 25) |
| | | AATTCAAACAGGCAGAAGCAGTGACCTCTCCGCGTC | (SEQ ID NO: 26) |
| | | GTTTGTCCGTCTTCGTCACTGGAGAGGCGCAGTTAA | (SEQ ID NO: 27) |
| 8 | IKK-I | D S G L D S M | (SEQ ID NO: 28) |
| | | AATTCGATAGCGGTCTGGATTCCATGC | (SEQ ID NO: 29) |
| | | GCTATCGCCAGACCTAAGGTACGTTAA | (SEQ ID NO: 30) |
| 9 | JNK3 | E L V E P L T P S G E A P N Q A | (SEQ ID NO: 31) |
| | | AATTCGAACTGGTGGAACCGCTGACTCCAAGCGGCGAAGCACCGAACCAGGCAC | (SEQ ID NO: 32) |
| | | GCTTGACCACCTTGGCGACTGAGGTTCGCCGCTTCGTGGCTTGGTCCGTGTTAA | (SEQ ID NO: 33) |
| 10 | PKA | A G G T G G S L R R A S L P G T G G S E L | (SEQ ID NO: 34) |
| | | AATTCGCAGGCGGTACCGGCGGCAGCCTGCGTCGCGCGAGTCTGCCGGGTACCGGTGGCAGCGAACTGC | (SEQ ID NO: 35) |
| | | GCGTCCGCCATGGCCGCCGTCGGACGCAGCGCGCTCAGACGGCCCATGGCCACCGTCGCTTGACGTTAA | (SEQ ID NO: 36) |
| 11 | AMPK | H M R S A M S G L H L Q F H M R S A M S | (SEQ ID NO: 37) |
| | | AATTCCATATGCGCAGTGCAATGTCTGGTCTGCATCTGCAATTCCATATGCGCAGTGCAATGTCG(N)₅₀C | (SEQ ID NO: 38) |
| | | GGTATACGCGTCACGTTACAGACCAGACGTAGACGTTAAGGTATACGCGTCACGTTACAGC(N)₅₀GTTAA | (SEQ ID NO: 39) |
| 12 | Src1 | E I Y G E F G | (SEQ ID NO: 40) |
| | | AATTCGAAACCTATGGCGAATTTGGCC | (SEQ ID NO: 41) |
| | | GCTTTGGATACCGCTTAAACCGGTTAA | (SEQ ID NO: 42) |

*In addition to the proteins listed in the table, GST fusion proteins substrates for following kinases have been generated: AMPKK, ATM, EF2K, GRK, FAK, PKC, PKR, SRK1.
Footnotes:
1. Yang F. et al., Anal Biochem. 15; 266(2): 167-73, 1999; proteins: GST-Ablx1, GST-Z2-Ablx1
2. Sanchez, Y. et al., Science 277: 1497-1501, 1997; Furnari B., et al., Science 277: 1495-1497, 1997. Seq from CDC25C, Ser 216 is target; proteins: GST-CHKx5, GST-CHKx9
3. Marin, O. et al., Biochem. Biophys. Res. Commun. 198: 898-905, 1994; proteins: GST-CK1x5
4. Kuenzel, E. A., et al., J. Biol. Chem. 262: 9136-9140, 1987; Litchfield, D. W., et al., J. Biol. Chem. 265: 7638-7644, 1990; proteins: GST-CK2x4
5. Lees-Miller SP, Anderson C W., J Biol Chem. 264(29): 17275-80, 1989. Ser/Thr kinase dependent on DNA; proteins: GST-Z1-dsDNAx4
6. Beebe J. A. et al., J. Biol. Chem., 278: 26810-26816, 2003; proteins: GST-EGFRx3
7. Lindgren N, et al., Eur J Neurosci. 2002 15(4): 769-73, 2002; Haycock J W. J Neurosci Methods. 2002 Apr 30; 116(1): 29-34. Corresponds to amino acids 24-33 of rat tyrosine hydroxylase; proteins: GST-ERKx2
8. Nandini Kishore N. et al., J. Biol. Chem., 277(16), 13840-13847, 2002; proteins: GST-IKKX4, GST-IKKx6
9. Schiaffonati L, et al., Neurosci Lett., 312(2):75-78, 2001; Nihalani, D., et al, J Biol Chem, 278: 28694-702, 2003; proteins: GST-JNK3x3, GST-JNK3x8
10. Zhang J. et al., Proc Natl Acad Sci U S A. 98(26): 4997-5002, 2001; proteins: GST-Z1-PKA
11. Davies S. P. et al., Eur. J. Biochem., 186: 123-128, 1989; Carling D., et al., Eur. J. Biochem., 186: 129-136, 1989; proteins: GST-Z2-AMPKx2
12. Yang F. et al., Anal Biochem., 266(2): 167-73, 1999; proteins: GST-Z2-Srcx3.

Each double-stranded DNA oligonucleotide started from a 5′-AATTC sequence, which generates an EcoRI site when the oligonucleotide is ligated to an EcoRI cut DNA fragment. Each double-stranded DNA oligonucleotide ends with a CTTAA-5′ sequence, which generates an incomplete EcoRI site (designated EcoRI*) when ligated to an EcoRI DNA fragment. The oligonucleotides encoded peptide substrate sequences in frame with the GST open reading frame when inserted into the EcoRI site of the pGEX-6p-1 vector, as well as in frame with each other in the case where concatamers of the dsDNA oligonucleotides are inserted into the site in correct orientation. A variation in the degree of concatamerization is desirable to provide for a variation in the substrate sizes. Furthermore, as will be discussed below, increasing the number of monomers in a concatamer can result in the increase of the activity of the substrate.

First, the pGEX-6p-1 vector was cleaved with EcoRI and AatII restriction endonucleases for 2 hours at 37° C. in a reaction mixture containing 100 μg/ml plasmid DNA, 500 units/ml EcoRI and 1000 units/ml AatII in NEB3×1 buffer (New England Biolabs). The EcoRI/AatII digestion generated two fragments: 302 base pairs (b.p.) and 4,682 b.p. The fragments were separated in 1.1% TBE-agarose gels and purified using QIAEX II Gel Extraction Kit (Qiagen). Next, the 302 b.p. fragment was ligated with a dsDNA oligonucleotides encoding protein kinase substrates (see FIG. 1, step A). The reaction mixtures contained 5 μg/ml of the 302 b.p. fragment, 100 μg/ml phosphorylated dsDNA oligonucleotides and 1000 units/ml T4 in 1×T4 DNA ligase reaction buffer (New England Biolabs). The reaction mixtures were incubated overnight at about 10° C., after which DNA was ethanol precipitated, dissolved in NEB3×1 buffer and double-digested with EcoRI and AatII restriction endonucleases (see FIG. 1, step B). The DNA was separated in 1.1% agarose gels and the 302 b.p. fragments ligated to different number of monomers of dsDNA oligonucleotides were purified from the gels.

Notably, this technique allows obtaining concatamers of the dsDNA oligonucleotides having correct orientation of monomers with respect to the EcoRI-AatII (302 b.p.) fragment. The monomers ligated in reverse orientation were cleaved from the fragment with EcoRI restriction endonuclease, since reverse orientation insertion generates a complete EcoRI site. On the contrary, ligation of monomers in the correct orientation generated an EcoRI* site, which is not recognized and cleaved by EcoRI. Therefore, monomers ligated in the correct orientation remain attached to the fragment after digestion with EcoRI.

The purified 302 b.p. fragments fused to correctly attached monomer(s) were ligated with equimolar amounts of the large (4,682 b.p.) pGEX-6p-1 EcoRI/AatII DNA fragment (see FIG. 1, step C). The reaction mixtures contained 5-10 μg/ml DNA and 100 units/ml T4 DNA ligase in the 1×T4 DNA ligase reaction buffer. The mixtures were incubated at 10° C. overnight and used to transform XL10-Gold ultracompetent cells (Stratagene) on agar plates with 100 μg/ml ampicillin.

Individual colonies were selected and incubated overnight in a 37° C. shaker in 5 ml of LB supplemented with 100 μg/ml ampicillin. Plasmid DNA was purified using the boiling method (Sambrook, J, Fritsch, E. F. and Maniatis, T. (1989) Molecular Cloning, A Laboratory Manual; Volume 3, Second edition. Cold Spring Harbor Laboratory). Plasmid DNA was digested with EcoRI and AatII as described above and the restriction fragments separated in 1.1% TBE agarose gels. The sizes of small EcoRI-AatII fragments were determined using 1 kb Plus DNA ladder (Invitrogen), which was run on the same gels. This allowed approximate determination of the number of monomers encoding protein kinase substrates in different clones. The DNA clones were also analyzed using double digestion with HincII and EcoRI or MscI and EcoRI restriction endonucleases. The restriction profiles were compared with the profiles of the parental vector pGEX-6p-1 digested using the same restriction endonucleases. HincII and MscI restriction endonucleases cleave in the GST gene sequence, therefore allowing detection of clones which have perturbation in the DNA sequence upstream of the EcoRI site. Such perturbations were rare, and such clones were discarded. The clones yielding expected restriction profiles were sequenced and used either for protein production and characterization (see FIG. 1, steps D and E) or for further manipulations to construct protein kinase substrates of a larger size.

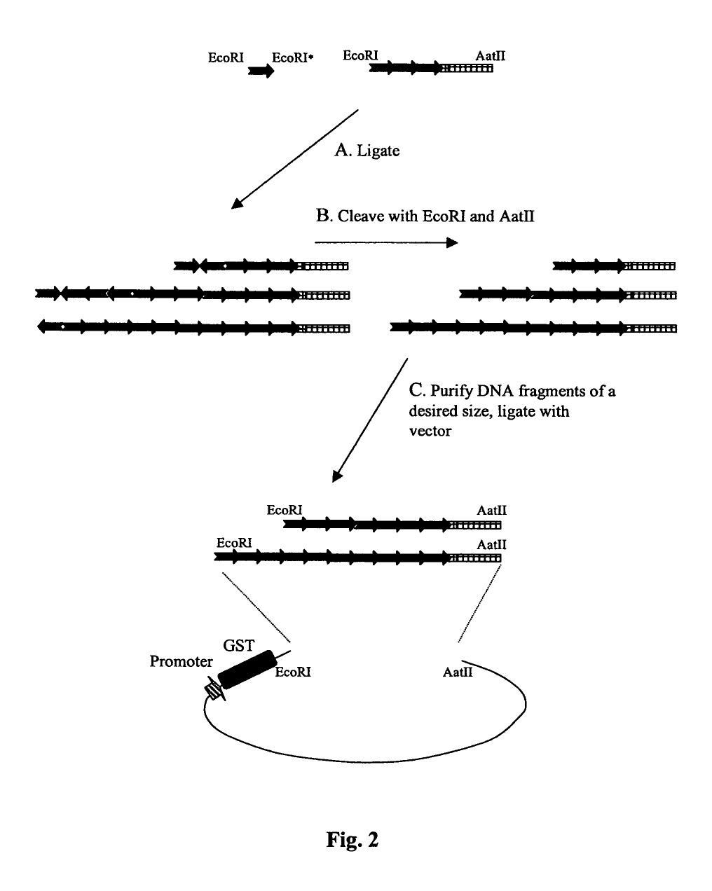

Conveniently, a modification of the standard scheme presented in FIG. 1 can be used to increase the number of monomers in a protein kinase substrate concatamer. In this case the manipulations are exactly the same as presented on FIG. 1, except at the initial step a small pGEX-6p-1 EcoRI-AatII 302 b.p. fragment is substituted for a small EcoRI-AatII fragment derived from a pGEX-6p-1-kinase substrate plasmid vector (FIG. 2).

### Example 2

### Plasmid Constructs Encoding Stuffing Fragments

Different numbers of monomers often provide satisfactory variation in size of the protein kinase or protein phosphatase substrates. However, in some cases it is desirable to provide a larger range of sizes of substrates, especially in the higher than 50 kDa molecular weight range, in order to be able to combine them into multiplex mixtures, which can be conveniently separated on SDS-PAGE gels. Furthermore, it has been found that some GST-protein kinase fusions can be insoluble under certain conditions. However, typically it is possible to recover sufficient quantities of soluble substrate when a stuffing fragment is incorporated into the fusion protein. To this end, DNA fragments encoding additional peptide sequences (stuffing fragments encoding stuffing peptide sequences) were inserted in-frame with the GST tag and with the protein kinase substrate to increase the size of the substrate fusion protein.

E. coli β-galactosidase was chosen as a source of the stuffing amino acid sequences because it is a large unphosphorylated protein that is soluble and can be expressed to a high level in E. coli. Furthermore, β-galactosidase is a multi-domain protein, which allows generation of stuffing fragments of different sizes through selection of domains and domain combinations. For stability reasons it is preferable to use whole proteins or protein domains as stuffing fragments, rather than randomly selected parts of the proteins, which could be problematic due to alterations in their folding. Three β-galactosidase regions were amplified using PCR and the pFR-βgal plasmid (Stratagene) as a template and the following pairs primers: Glycosyl hydrolases family 2, sugar binding domain (37 kd), designated Z2:

| | |
|---|---|
| Forward primer: | |
| GCGCGGATCCGGTGGCAGCTGGCGTAATAGCGAAGA | (SEQ ID NO: 1) |
| Reverse primer: | |
| GCGCGAATTCGCCTTTATGCAGCAACGAGACGTCA | (SEQ ID NO: 2) |

Glycosyl hydrolases family 2, sugar binding and immunoglobulin-like beta-sandwich domain domains (21 kd), designated Z1:

| | |
|---|---|
| Forward primer: | |
| GCGCGGATCCGGACAACGTCGTGACTGGGAAAA | (SEQ ID NO: 3) |
| Reverse primer: | |
| GCGCGAATTCACCATTTTCAATCCGCACCT | (SEQ ID NO: 4) |

Beta galactosidase small chain, N and C-terminal domains (32 kd), designated Z3:

| | | |
|---|---|---|
| Forward Primer: | GCGCGGATCCGGTCACGCCATCCCGCATCTGA | (SEQ ID NO:5) |
| Reverse Primer: | GCGCGAATTCGCCTTGACACCAGACCAACTGGTA | (SEQ ID NO:6). |

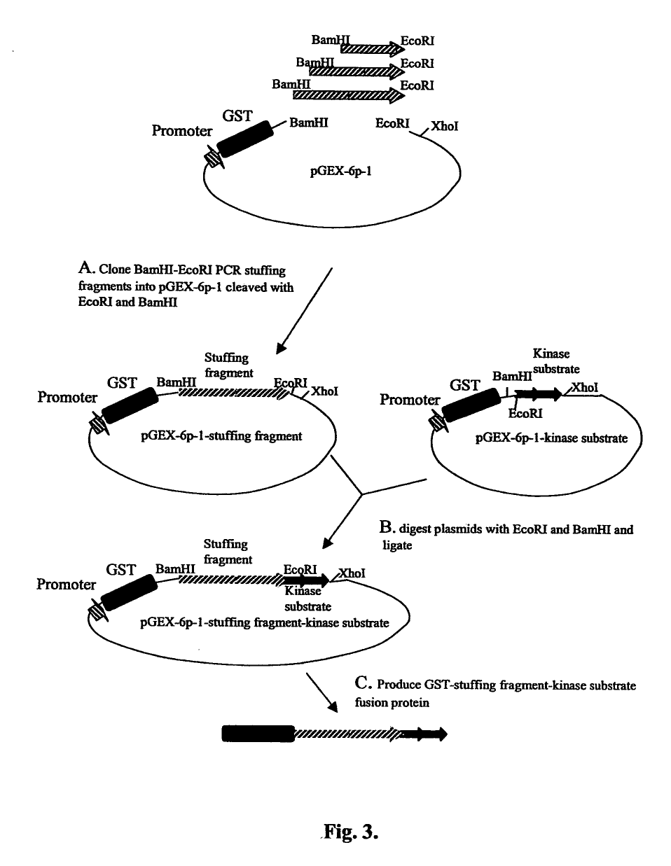

Each forward primer had a BamHI site (underlined) and each reverse primer had an EcoRI site (underlined). This allowed digestion of the PCR fragment at the ends with BamHI and EcoRI restriction endonucleases and cloning them into pGEX-6p-1 plasmid vector (see FIG. 3, step A). To combine the stuffing fragments with protein kinase substrates, the plasmids with stuffing fragment inserts were cleaved with PstI and EcoRI restriction endonucleases in EcoRI buffer (New England Biolabs), which yielded large (over 4 kb) and small (under 1.5 kb) DNA fragments. Large PstI-EcoRI fragments encoding the GST moiety and a stuffing fragment moiety were purified after separation in 1% agarose gels. Similarly, plasmids encoding kinase substrate peptides were cleaved using the same restriction endonucleases and small EcoRI-PstI fragments encoding a protein kinase substrate were purified. Large fragments from a pGEX-6p-1-stuffing plasmids and small fragments from pGEX-6p-1-kinase substrate plasmids were ligated using T4 DNA ligase and the ligation mixture was transformed into XL10-Gold ultracompetent cells (Stratagene) on agar plates with 100 ug/ml ampicillin.

Resulting ampicillin-resistant colonies were used to purify plasmid DNA as described above. They were digested with EcoRI and PstI or with EcoRI and BamHI restriction endonucleases and the DNA fragments were separated in 1% agarose gels. Clones yielding the fragment sizes indicative of DNA inserts encoding protein kinase substrates and stuffing fragments were sequenced and selected for further work to produce GST-stuffing-kinase substrate fusion proteins (see FIG. 3, step C).

### Example 3

### Production of GST Fusion Kinase Substrates

GST fusion protein kinase substrates were produced as originally described for production of GST fusion proteins with minor modifications (Smith DB and Johnson K S, Single-step purification of polypeptides expressed in E. coli as fusions with glutathione S-transferase. Gene, 67: 31-40, 1988).

In brief, overnight cultures of XL10-Gold E. coli transformed with plasmid vectors providing for expression of GST-tagged protein kinase substrates were diluted 1:10 in 250 ml of fresh LB medium and grown for 1 h at 37° C. before adding IPTG (Stratagene) to 0.1 mM. After further 3 hours of growth, cells were pelleted and resuspended in 1/50 volume of PBS, and protease inhibitors cocktail (Roche Diagnostics Gmbh, Cat #1 697 498) was added according to the manufacturer's instructions. Cells were sonicated at mild sonication using Branson 250 Sonifier at output 40 on ice for 0.5 min, after which Triton X-100 was added to 1% concentration and the sonication step was repeated. The insoluble fraction was removed by centrifugation for 20 min at 16,000 rpm at 4° C. in SA-600 rotor using Sorvall RC 5C centrifuge. The supernatant was mixed with 1/10 volume of 50% slurry of Glutathione Sepharose™ 4B in PBS (Amersham Biosciences AB, Cat #17-0756-01). The GST fusion proteins were adsorbed by rotation overnight at 5° C. and the beads were washed 3 times with ice-cold PBS (each wash about 15 times volume of the beads).

The GST fusion proteins were either eluted from the beads using 10 mM reduced glutathione in 50 mM Tris-HCl pH 8.0 or were left on the beads depending on the subsequent applications. In either case, the proteins were typically diluted to 1 mg/ml concentration and NaCl was added to 150 mM concentration, after which sterile glycerol was added to 20% vol/vol concentration and the proteins were stored at −20° C. Protein concentration was measured using Bradford reagent (Pierce).

### Example 4

### Glutathione Agarose Beads do not Interfere with the Assay

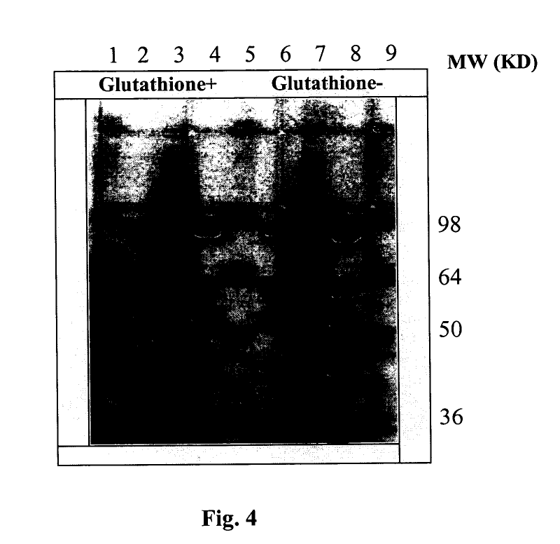

Providing kinase substrates on beads rather than in solution allows simple purification of the substrates after subjecting them to phosphorylation in complex protein mixtures, such as clarified crude cell extracts. GST-tagged soluble protein kinase substrates can be also applied for the same purpose. However, in this case it is often preferable to re-capture them to the glutathione beads in order to purify them from a complex protein mixture. In general, recapturing substrates on the beads can be achieved with about 50% or more recovery with recapture times of about 2 hours. FIG. 4 shows data supporting this conclusion.

More specifically, FIG. 4 shows recovery of soluble substrate ladder from crude cell extracts. HeLa cells growing to confluency in 75 cm² flasks were harvested into 1 ml of ladder buffer (40 mM MOPS pH 7.0, 1 mM EDTA) and sonicated for 30 seconds in a water bath sonicator. The cell lysates were centrifuged for 5 min at 14,000 rpm, and the insoluble and soluble crude cell lysate fractions were separated. Thirty (30) μl of the soluble fraction was mixed with 5 μl of a soluble pre-mixed five-member protein ladder (GST-Z1-dsDNA; GST-JNK3×6; GST-CHK×8; GST-IKK×6; GST-EGFR×3; nomenclature indicates identity of the substrates used, not kinases, and the indication “×6”, “×8”, and “×3” indicates the number of target site repeats) containing 50 mM Tris-HCl pH 8.0, 150 mM NaCl, and 1 mg/ml total protein.

In order to establish if glutathione might interfere with the adsorption of the GST-tagged substrates to the beads, another experiment was performed with the same ladder, but containing 10 mM glutathione. Ten (10) μl of a 50% slurry of Glutathione Sepharose 4B beads in PBS (Amersham Biosciences AB) was added to the mixture and incubated at 5° C. on a rotator at 40 rpm for 2 hours. The protein kinase substrates attached to the beads were purified from the reaction mixtures using centrifugation (10,000 rpm, 1 min). The beads were washed three times in 200 μl PBS and pelleted. The beads were resuspended in 20 μl of 1×NuPAGE loading buffer with a reducing agent, heated for 1 min at 100° C. and the proteins were separated in 10% SDS-PAGE. The lanes shown in FIG. 4 contained the following: Lanes 1, 5, 9—molecular weight marker; Lanes 2,6—GST tagged five-member protein ladder recovered from crude cell lysates on Glutathione Sepharose beads; Lanes 4, 8-five-member protein ladder before adding to the cell lysates. The samples volumes were normalized, i.e., the samples on lanes 2, 4, 6, 8 would look identical if the ladder recovery were 100%.

Recapturing requires additional incubation time of the substrates with the protein mixtures, which is undesirable if proteolytic degradation is an issue. However, this potential problem can be addressed by inclusion of proteinase inhibitors or performing recapture at low temperatures. Furthermore, the yield of the proteins on the beads is typically on the order of 2 times or greater higher than the yield of the same protein eluted from the beads, since it is sometimes difficult to completely elute the protein. Moreover, if a protein kinase substrate is available in low amounts, it might be preferable to bind it to a solid support initially to minimize losses often seen with recapturing.

Although attaching substrates to beads has its advantages, for some applications (for example characterization of purified kinases), there is no apparent significant differences between soluble and on-the-beads formats. Some users might prefer soluble format as a personal preference, for example due to easier pipetting.

### Example 5

### Characterization of Certain Protein Kinase Substrates

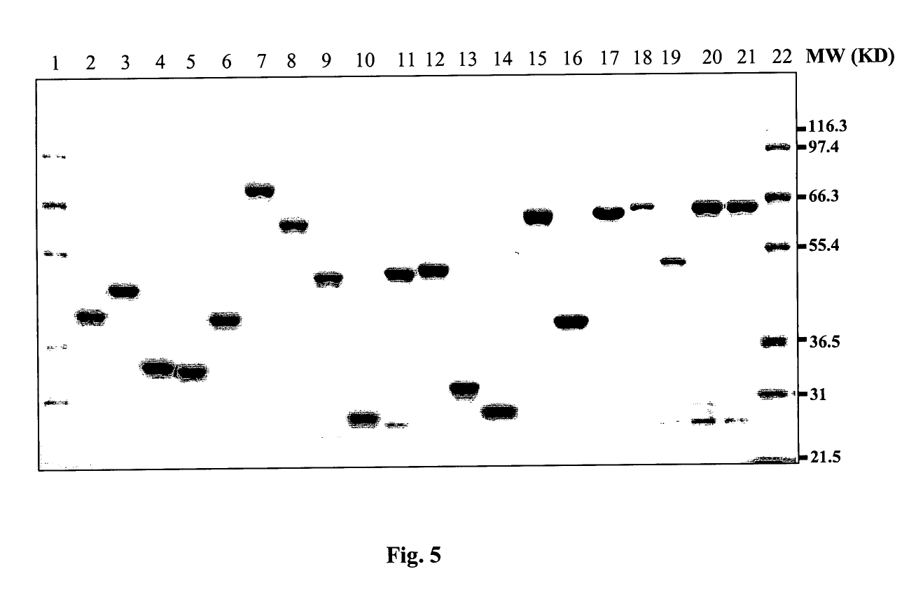

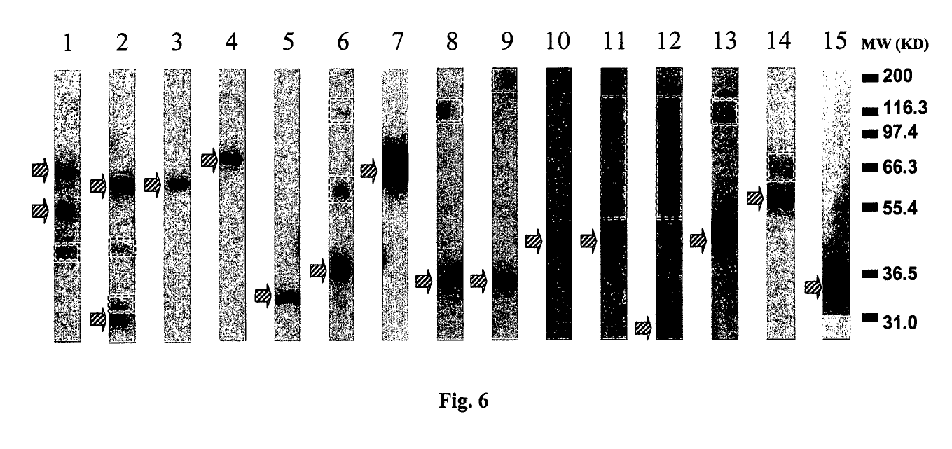

Protein kinase substrates were produced in on-the-beads format as described above and investigated in SDS-PAGE and for their purity and characteristic mobility in the SDS-PAGE (see FIG. 5) as well as for their specific activity with corresponding protein kinases (see FIG. 6).

FIG. 5 shows the results of an SDS-PAGE of protein kinase substrates stained with Coomassie Blue. GST fusion protein kinase substrates were purified using affinity chromatography on Glutathione Sepharose 4B beads (Amersham). The substrates were stored frozen at −20° C. on the beads in 40 mM Tris-HCl pH 8.0, 125 mM NaCl, 20% glycerol. Two (2) μg of a substrate was loaded on each lane of TBE-10% polyacrylamide gels except lanes 18 and 19, where 0.25 μg was loaded. The lanes shown in FIG. 5 contained the following: Lanes 1, 22—Mark 12 molecular weight marker (Invitrogen); Lane 2—GST-CHK×5; Lane 3—GST-CK1×5; Lane 4—GST-CK2×4; Lane 5—GST-IKK×4; Lane 6—GST-JNK3×3; Lane 7—GST-Z1-dsDNA×4; Lane 8—GST-Z1-PKA; Lane 9—GST-Z2-Src×2; Lane 10—GST-Abl×1; Lane 11—GST-Z2-Abl×1; Lane 12—GST-CHK×9; Lane 13—GST-EGFR×3; Lane 14—GST-ERK×2; Lane 15—GST-Z1-ERK×2; Lane 16—GST-IKK×6; Lane 17—GST-JNK3×8; Lane 18—GST-Z1-AMPK×2; Lane 19—GST-Z2-AMPK×2; Lanes 20, 21—GST-Z1-AMPK×2. As with FIG. 4, the nomenclature indicates identity of the substrates used, not kinases, and the indication “×3”, “×4”, “×5”, etc. indicates the number of target site repeats.

FIG. 6 shows phosphorylation of protein kinase substrates. To investigate the specific activity, 2 μl of protein kinase substrate attached to the beads (1 μg) was mixed with 10-500 units of appropriate protein kinase, ³²P-ATP and 2.5-20 μl of appropriate reaction buffer provided by the manufacturer. The reaction mixture was incubated for 0.5 hours at 37° C. on a shaking platform at 50 r.p.m. and the proteins were separated in 10% SDS-PAGE. The gels were dried and exposed with BioMax MR film (Kodak). The film was developed after 1 hour exposure, and radioactive phosphorylated substrates were detected on the autoradiogram as bands corresponding to the substrates' molecular weights.

Phosphorylated protein kinase substrates are indicated by arrows, bands corresponding to autophosphorylated protein kinases are indicated by boxes. The lanes shown in FIG. 6 contained the following: Lane 1—GST-Z1-AMPK×3 (upper band) and GST-Z2-AMPK×3 (lower band); Lane 2—GST-Z1-ERK×2 (upper band) and GST-ERK×2 (lower band); Lane 3—GST-Z1-PKA; Lane 4—GST-Z1-dsDNA×4; Lane 5—GST-CK2×4; Lane 6—GST-JNK3×3; Lane 7—GST-JNK3×8; Lane 8—GST-EGFR×3; Lane 9—GST-IKK×4; Lane 10—GST-IKK×7; Lane 11—GST-Z2-Abl×1; Lane 12—GST-Abl×1; Lane 13—GST-CK1×4; Lane 14—GST—Z2-Src×2; Lane 15—GST-CHK×5. As with FIGS. 4 and 5, the nomenclature indicates identity of the substrates used, not kinases, and the indication “×4”, etc. indicates the number of target site repeats.

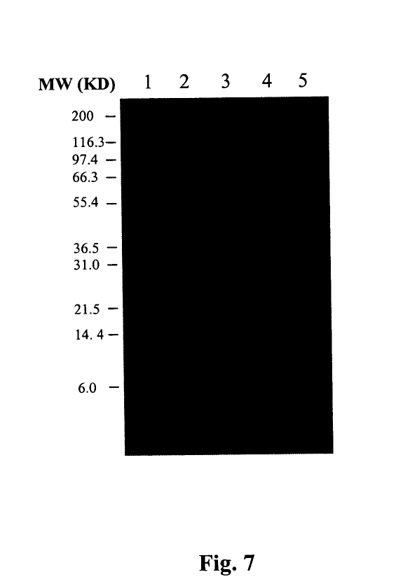

FIG. 7 shows that an increase in substrate reactivity can be contingent upon the number of the substrate peptide repeats in the GST fusion proteins. The figure is an autoradiogram of phosphorylated GST-dsDNA kinase peptide substrate fusion proteins with different number of the peptide repeats. The lanes shown in FIG. 7 contained: Lane 1—GST-dsDNA×1; Lane 2—GST-dsDNA×2; Lane 3—GST-dsDNA×3; Lane 4—GST-dsDNA×4, Lane 5—no substrate (negative control). The fusion proteins were phosphorylated in reaction mixtures containing 10 μl of 5×DNA PK reaction buffer (250 mM HEPES, pH 7.5, 500 mM KCl, 50 mM MgCl₂, 1 mM EGTA, 0.5 mM EDTA, 5 mM dithiothereitol), 5 μl of 100 μg/ml solution of thymus DNA, 2 μl of 2 mg/ml solution of BSA, 1 μg dsDNA PK substrate, 2 μl³²-P ATP (10 μCi/μl), 26 μl H₂O, 1 μl dsDNA PK (100 units) (Promega). The reaction mixtures were incubated at 37° C. for 1 hour, the proteins were separated in 4-20% SDS-PAGE and exposed to BioMax MR film (Kodak).

To investigate the specific activity, 2 μl of protein kinase substrate (from about 0.5 to about 1 μg) was mixed with 10-500 units of appropriate protein kinase, 2 μl of ³²P ATP (10 μCi/μl) and the mix was brought up to 25 μl in the appropriate reaction buffer provided by the manufacturer. The reaction mixture was incubated for 0.5 hour at 37° C. on a shaking platform at 50 r.p.m. Fifteen (15) μl of 3× sample buffer with reducing agent was added to the reactions, the samples were heated to 100° C. for 1 min, and ½ of the sample was separated using SDS-PAGE on 10% Tris-Glycine gels. A number of experiments have been done in which the shaking was omitted, and no appreciable difference in the reaction was observed (data not shown).

After staining with Coomassie Blue and destaining, the gels were dried and exposed to BioMax MR film (Kodak). The film was developed after 1 hour exposure, and radioactive phosphorylated substrates were detected on the autoradiogram as bands corresponding to the substrate's molecular weights. All of the protein kinase substrates reacted with their corresponding kinases (see FIG. 6). As expected, the reactivity of different substrates varied. Some substrates appeared overexposed, and some appeared underexposed when processed in similar conditions. The reactivity of the substrates can generally be increased by increasing the number of the substrate monomer repeats in a concatamer (see FIG. 7). Signals of substrates can also be increased by loading more substrate in a gel lane (data not shown).

As expected, the substrates exhibited the strongest reactivity with their specific kinases. Nevertheless, some cross-reactivity was observed, summarized in Table 2. This cross-reactivity can be accounted for, and conclusions drawn as to the presence of a particular protein kinase or a mixture of two or more protein kinases. Likewise, it can be accounted for, and a conclusion drawn that at least one of a limited number of cross-reacting kinases are present in the sample tested.

TABLE 2
Reactivity for each of the Kinase Substrates Available

| Kinase | | | | | dsDNA | | | | | | | |
|---|---|---|---|---|---|---|---|---|---|---|---|---|
| Substrate | Abl | CHK | CK1 | CK2 | (DNAPK) | EGFR | JNK3 | IKK | MAPK | AMPK | Src | PKA |
| Abl | +++++ | | | | | +++++ | | | +++++ | | +++++ | |
| CHK | | +++++ | | | | | | | | | | |
| CK1 | | | +++++ | | | | | +++++ | +++++ | | +++++ | |
| CK2 | | | | +++++ | | | | | | | | |
| dsDNAPK | | | +++++ | | +++++ | | | | | | | +++++ |
| EGFR | +++++ | | | | | +++++ | | | | | | |
| JNK3 | | | | | | | +++++ | + | | | + | |
| IKK | | | + | + | | | | +++++ | +++++ | | | |
| ERK | | | | | | | + | + | +++++ | | | |
| AMPK | +++ | | | | | | | | | +++++ | +++ | |
| Src | | | +++++ | | | | | + | | | +++++ | +++++ |
| PKA | | | | | +++++ | | | | | | | +++++ |

As an alternative to the radioactivity methods, a number of non-radioactive methods can be used, such as the ones employing phosphoprotein specific antibodies and phosphoprotein specific dies. Though using radioactivity is not always convenient, non-radioactive methods are often less sensitive and not as universal as the radioactivity methods. Studies have shown that good results can be obtained using GelCode phosphoprotein staining kit available from Pierce (Cat# 24550). It is suitable for detection of phosphoserine and phosphothreonine, but not for detection of phosphotyrosine. Molecular Probes offers the Pro-Q Diamond fluorescent stain that is suitable, based on information provided by the manufacturer.

### Example 6

### Measuring Concentration of GST Fusion Proteins Attached to Beads

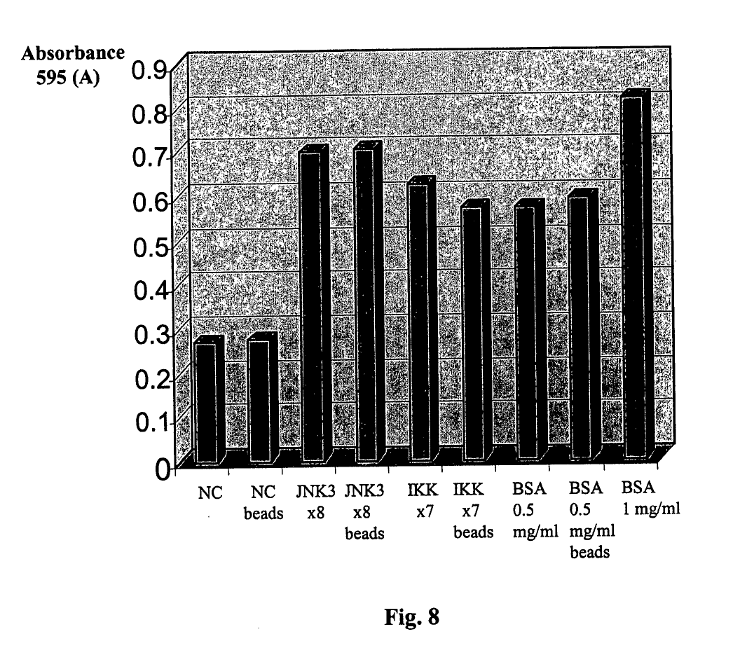

It is often important to be able to measure the concentration of proteins attached to the beads in the beads slurry in order to standardize the products and reproduce the experimental data. Concentration of a GST fusion protein in the beads slurry can be conveniently measured with the Coomassie dye-based Bradford assay (Pierce, Cat# 23238). It was found that beads do not significantly interfere with the assay, and the measurements obtained with GST fusion proteins combined with the beads were very similar to the measurements obtained with the same proteins measured in solution without the beads (see FIG. 8).

FIG. 8 shows a Bradford assay performed with soluble proteins and the same proteins attached to the beads. Five (5) μl of GST-JNK3×8 or GST-IKK×7 protein preparation was captured to 5 μl of 50% of glutathione sepharose slurry in 50 mM Tris-HCl pH 8.0, 150 mM NaCl. The negative control (NC) (5 μl of 50 mM Tris-HCl pH 8.0, 150 mM NaCl buffer) or 0.5 mg/ml solution of bovine serum albumin (BSA) was also spiked with the beads. 200 μl of Bradford reagent was added to each sample and absorbance at 595 nm was measured. The figure shows the average of 2 measurements.

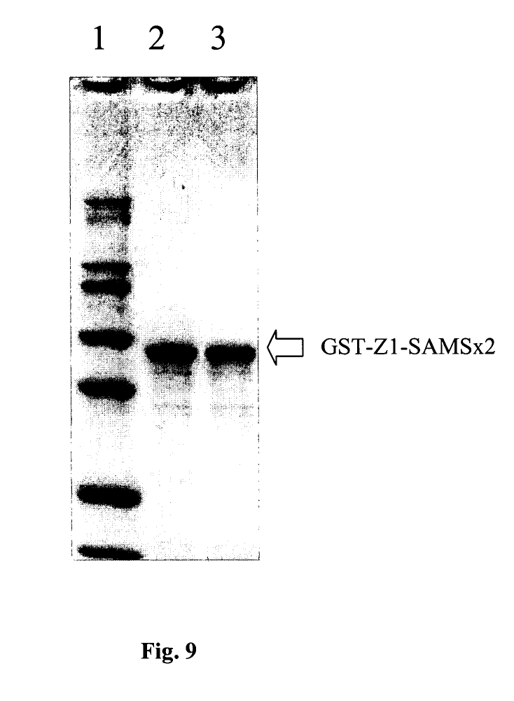

Moreover, proteins on the beads or in solution which were adjusted to the same concentration based on the Bradford assay measurement, were apparently the same concentration when analyzed on Coomassie Blue stained SDS-PAGE gels (see FIG. 9).

FIG. 9 shows the similarity of band intensity on Coomassie Blue stained gels of soluble and on-the beads protein preparations adjusted to the same concentration based on Bradford assay. GST-Z1-AMPK×2 protein concentration was measured using the Bradford assay while it was attached to the glutathione sepharose beads or after elution from the beads. Protein concentration in both samples was adjusted to 0.32 mg/ml. 8 μl of each protein sample was separated in 10% SDS-PAGE and stained with Coomassie Blue. The lanes shown in the figure contain: Lane 1-molecular weight marker (Mark 12, Invitrogen); Lane 2—GST-Z1-AMPK×2 on the beads; Lane 3—GST-Z1-AMPK×2 in solution.

### Example 7

### Freezing Substrates Attached to the Beads

Protein kinase substrates are typically stored frozen in 20% glycerol, at −20° C. Therefore, it is logical to apply these storage conditions to the protein kinase substrates which are attached to the beads. However, initially it was not clear if freezing the beads could compromise their structural integrity, affect the attachment of the substrates to the beads, or alter the reactivity of the substrates with protein kinases. To investigate potential effect of freezing-thawing, GST-IKK×4 protein kinase substrate attached to the beads was adjusted to 4 mg/ml protein concentration. Such a large amount of protein was used with the purpose to detect any signs of protein degradation or small amount of protein if it is coming off the beads after the freeze-thaw cycles.

A 50% slurry of glutathione-sepharose beads in 40 mM Tris-HCl pH 8.0, 120 mM NaCl, 20% glycerol with adsorbed protein kinase substrate GST-IKK×4, was subjected to five rapid freeze-thaw cycles using dry ice ethanol bath and 37° C. water bath. Aliquots of the slurry were removed before each freeze-thaw cycle and after the final one. The remaining beads were centrifuged in a microfuge at 2,000 rpm for 2 minutes and an aliquot of the supernatant was removed. Aliquots (5 μl) of both beads and supernatant were subjected to kinase reactions and one quarter of the reaction-mix was loaded on a 10% Tris-Glycine gel.

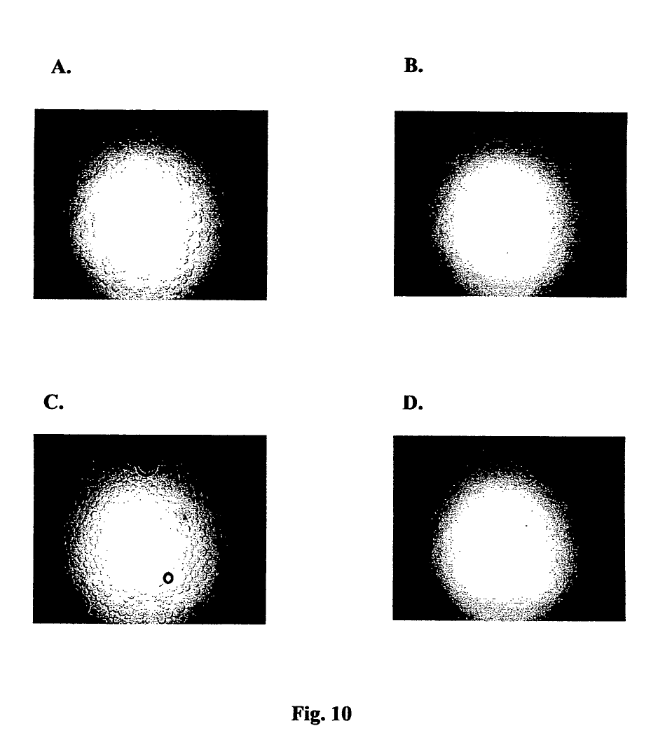

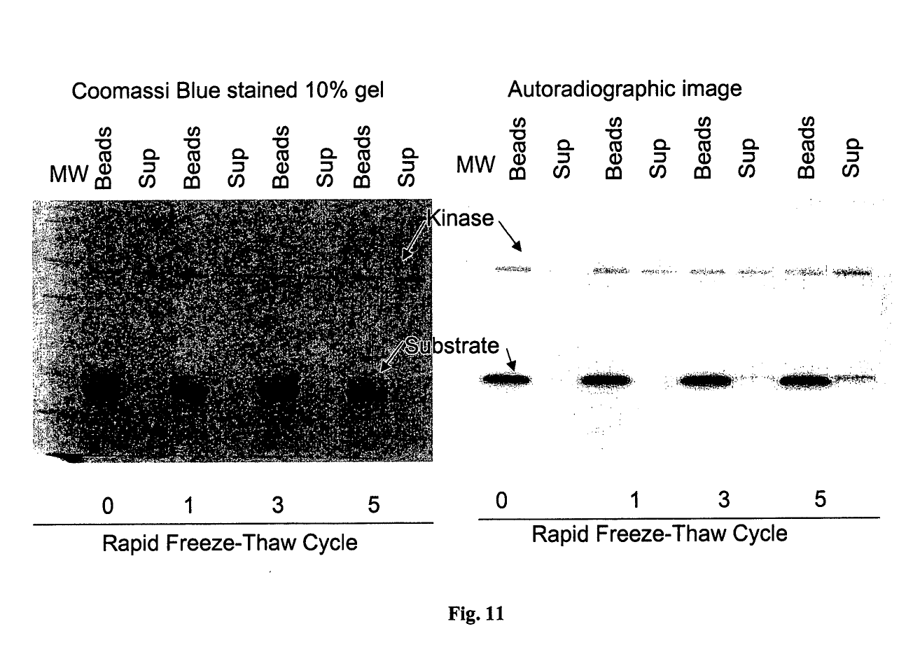

Apparently, freezing the beads did not compromise their integrity, as they looked exactly the same under the microscope before or after the freezing (see FIG. 10, which depicts pictures of the beads and supernatant under the microscope). Furthermore, the substrate was active and the vast majority remained attached to the beads despite the freezing. The freezing did not result in the substrate degradation (see FIG. 11). FIG. 11 shows that, after 5 freeze-thaw cycles in rapid succession only a minor amount of protein was in the supernatant. It appears that most protein that might come off beads during rapid freeze-thaw experiments likely re-binds to the beads upon further incubation.

FIG. 10 shows that the integrity of glutathione sepharose beads after freeze-thaw cycles remains. A 50% slurry of glutathione-sepharose beads in 40 mM Tris-HCl pH 8.0, 120 mM NaCl, 20% glycerol with adsorbed protein kinase substrate GST-IKK×1, was subjected to five freeze-thaw cycles using dry ice ethanol bath and 37° C. water bath. Aliquots of the slurry before and after the freeze-thaw cycles were centrifuged in a microfuge at 2,000 rpm for 2 minutes and the beads and the supernatants were examined under the light microscope at 40× magnification. FIG. 11 shows the results of freeze-thaw cycles on the GST-IKK×4 on beads, and shows that little or no substrate is lost from the beads, even after five freeze-thaw cycles.

### Example 8

### Multiplexing Individual Substrates into a Protein Kinase Substrate Ladder

Protein kinase substrates of different sizes and specific for different protein kinase activities can be combined into mixtures (protein kinase ladders). Combining several substrates (multiplexing) is convenient, as activation or repression of several kinase activities can be simultaneously followed in the same sample. A user can mix and match his own protein kinase ladders depending upon specific interest in activation of particular cell signalling pathways.

As an example, we combined GST-EGFR×3 (32 kDa), GST-IKK×7 (40 kDa), GST-Z2-Src×2 (49 kDa), and GST-JNK3×8 (61 kDa) protein kinase substrates into a 4-member ladder. The substrates were attached to the beads and the concentration of each substrate in the bead slurry was 0.2 mg/ml.

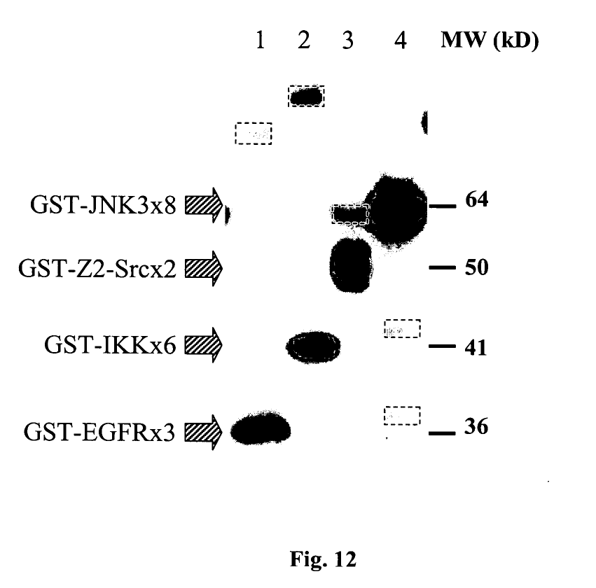

To investigate the performance of the ladder, 2 μl of protein kinase substrate (0.5-1 μg) was mixed with 10-500 units of appropriate protein kinase, 2 μl of ³²P-ATP (10 μCi/μl) and the mix was brought up to 25 μl in the appropriate reaction buffer provided by the manufacturer. The reaction mixture was incubated for 0.5 hours at 37° C. on a shaking platform at 50 r.p.m. and the proteins were separated in SDS-PAGE on 10% Tris-Glycine gels. After staining with Coomassie Blue and destaining, the gels were dried and exposed to BioMax MR film (Kodak). The film was developed after 1 hour exposure, and radioactive phosphorylated substrates were detected on the autoradiogram as bands corresponding to the substrates' molecular weights. All of the protein kinase substrates reacted with their corresponding kinases, and cross-reactivity with other substrate was minimal (see FIG. 12).

FIG. 12 shows phosphorylation of 4-member protein kinase ladder with protein kinases specific to the ladder components. Two (2) μl of protein kinase substrate (1 μg) was mixed with 10-500 units of appropriate protein kinase, 2 μl of ³²P-ATP (10 μCi/μl) and 25 μl of the ladder assay buffer (40 mM MOPS pH 7.0, 1 mM EDTA). The reaction mixture was incubated for 0.5 hours at 37° C. on a shaking platform at 50 r.p.m. and the proteins were separated in SDS-PAGE on 10% Tris-Glycine gels. The gels were dried and exposed to BioMax MR film (Kodak). The film was developed after 1 hour exposure, and radioactive phosphorylated substrates were detected on the autoradiogram as bands corresponding to the substrates' molecular weights. Phosphorylated protein kinase substrates are indicated by arrows, and bands corresponding to autophosphorylated protein kinases are indicated by boxes. The lanes shown in the figure contain: Lane 1—phosphorylation by EGFR kinase; Lane 2—phosphorylation by IKK; Lane 3—phosphorylation by Src kinase; Lane 4—phosphorylation by JNK3.

### Example 9

### Reaction Conditions

The following is a standard protocol that can be used as an assay according to the invention. Kits of the invention may contain some or all of the components, reagents, substrates (at least two substrates, having different molecular weights, different tags, or different protein kinase specificities) described in this example.

Prepare negative control and experimental reactions by adding the following components to separate microcentrifuge tubes in order:
Negative Control Reaction
   ≧2.0 μl of kinase substrate or 4 μl of substrate ladder
   5.0 μl of reaction buffer (5×)
   2.0 μl of [γ³²-P]ATP (100 μM) or 2.0 μl of Magnesium/ATP Cocktail
   X μl dH₂O (to a final volume of 25 μl)
Experimental Reaction
   X μl of kinase substrate or substrate ladder (equal amount to Negative Control Reaction)
   5.0 μl of reaction buffer (5×)
   2.0 μl of [γ³²P]ATP (100 μM) or 2.0 μl of Magnesium/ATP Cocktail
   X μg of pure kinase or experimental lysate
   X μl dH₂O (to a final volume of 25 μl)

Incubate reactions at 37° C. for 30 minutes with gentle shaking.

Optionally wash the reactions by adding 200 μl of PBS to the reaction mixtures and mix. Pellet the beads by spinning 1 minute in a microfuge at 2,000×g.

Remove the wash carefully, leaving the bead pellet in a few microliters at the bottom of the tube.

Wash again with 200 μl of PBS and transfer the beads and wash to a new tube before pelletting. Repeat the previous step in the new tube.

Optionally, repeat the wash, for example, if background noise is a problem.

Perform SDS-PAGE analysis of kinase phosphorylation. Note that this particular protocol is applicable for radioactive, colorimetric and fluorescence detection methods of substrate phosphorylation.

If beads were not washed, add 15 μl of 3×SDS-PAGE sample buffer containing reducing agent (10% w/v electrophoresis grade SDS, 20% v/v glycerol, 0.1% w/v bromophenol blue, 25% 0.5 M Tris-HCl, pH 6.8, 2-5% v/v BME, dissolved in water) to each reaction sample and mix. If beads were washed, then add an appropriate amount of SDS-PAGE sample buffer containing reducing agent, depending on the residual volume. It is preferable that the reducing agent is active

Boil samples for 1 minute and spin briefly to pellet beads

Samples can now be stored at −20° C. if needed. Frozen samples should be reheated prior to SDS-PAGE analysis.

Load 15-20 μl (or about ½) of each reaction sample onto a 10% Tris-Glycine polyacrylamide gel. Loading beads into the well should be avoided.

Electrophorese the gel until the dye front has run off of the bottom edge of the gel.

Stain the gel in Coomassie Blue or another suitable stain, such as silver stain.

Destain the gel completely.

Rinse the gel in water for 5 minutes.

Radioactive detection of kinase phosphorylation:
dry the gel
expose the dried gel overnight against autoradiographic film at room temperature (note: if purified kinase was used, exposure can be as short as 30 minutes or less)

Colorimetric and fluorescence detection of kinase phosphorylation:
dye the gel using a commercially available phosphoprotein staining kit following the manufacturer's instructions.

It will be apparent to those skilled in the art that various modifications and variations can be made in the practice of the present invention without departing from the scope or spirit of the invention. In addition, it will be apparent to those of skill in the art that the presently disclosed concepts can be applied to protein kinase recognition/phosphorylation sites that are discovered after issuance of this patent, and that application of these concepts to those target sites will result in substrates, nucleic acids, compositions, assays, and kits that are envisioned by this invention. Other embodiments of the invention will be apparent to those skilled in the art from consideration of the specification and practice of the invention. It is intended that the specification and examples be considered as exemplary only, with a true scope and spirit of the invention being indicated by the following claims.

1. A composition comprising at least two different protein kinase or protein phosphatase substrates, said substrates independently comprising a tag and at least one target site specific for a protein kinase or protein phosphatase,
wherein all target sites present on one substrate are specific for the same protein kinase or protein phosphatase,
wherein each substrate is specific for a different protein kinase or set of protein kinases that share a common target site, or specific for a different protein phosphatase or set of protein phosphatases that share a common target site, and
wherein each substrate differs in size from every other substrate or each substrate that is specific for a particular kinase or phosphatase has a different tag from all other substrates.

2. The composition of claim 1, wherein the tag is glutathione-S-transferase, biotin, or hexahistidine.

3. The composition of claim 1, wherein each substrate independently comprises between 1 and 9 target sites.

4. The composition of claim 1, wherein the target sites specific for each protein kinase or each protein phosphatase are different sizes.

5. The composition of claim 1, further comprising a solid support comprising a binding partner for the tag.

6. The composition of claim 1, wherein the substrates are independently specific for the following protein kinases: CHK, CK1, CK2, EGFR, ERK, IKK, JNK, MAPK, JNK3, PKA, AMPK, DNA-PK, EF2K, FAK, GRK, PKC, CDK1, ATM, Abl, and Src.

7. A nucleic acid encoding at least two different protein kinase or protein phosphatase substrates,
wherein the encoded substrates each comprise a tag and at least one target site specific for a protein kinase or a protein kinase,
wherein all target sites present on one substrate are specific for the same protein kinase or the same protein phosphatase,
wherein each substrate is specific for a different protein kinase or set of protein kinases that share a common target site, or specific for a different protein phosphatase or set of protein phosphatases that share a common target site, and
wherein each substrate differs in size from every other substrate.

8. The nucleic acid of claim 7, wherein expression of each encoded substrate is controlled independently of each other encoded substrate.

9. A cell comprising the nucleic acid of claim 7.

10. A composition comprising at least two different nucleic acids encoding protein kinase or protein phosphatase substrates,
wherein the nucleic acids independently encode different substrates comprising a tag and at least one target site specific for a protein kinase or a protein phosphatase,
wherein all target sites present on one encoded substrate are specific for the same protein kinase or protein phosphatase,
wherein each encoded substrate is specific for a different protein kinase or set of protein kinases that share a common target site, or specific for a different protein phosphatase or set of protein phosphatases that share a common target site, and
wherein each encoded substrate differs in size from every other substrate encoded by nucleic acids in the composition, or each encoded substrate that is specific for a particular kinase or phosphatase has a different tag from all other substrates encoded by nucleic acids in the composition.

11. The composition of claim 10, wherein the composition comprises at least one cell lysate.

12. A method of detecting the presence of a protein kinase, said method comprising
providing at least two different protein kinase substrates, said substrates independently comprising a tag and at least one target site specific for a protein kinase,
wherein all target sites present on one substrate are specific for the same protein kinase, and
wherein each substrate differs in size from every other substrate or wherein each substrate that is specific for a protein kinase or group of kinases that share a common target site comprises a different tag from every substrate that is specific for a different protein kinase or group of kinases that share a common target site;
providing a sample that contains or is suspected of containing a protein kinase;
combining the substrate and sample for an amount of time sufficient for a protein kinase to modify a substrate; and
determining if a substrate has been modified,
wherein the presence of a modified substrate indicates the presence of at least one protein kinase that specifically modifies the substrate.

13. The method of claim 12, wherein the combining is performed in vitro.

14. The method of claim 12, wherein the combining is performed in vivo.

15. The method of claim 12, wherein four different protein kinase substrates are provided, one specific for each of EGFR, IKK, JNK3, and Src.

16. The method of claim 12, wherein each substrate is provided in an amount of about 0.1 μg to about 1.0 μg.

17. The method of claim 12, wherein each substrate is bound to a solid support.

18. The method of claim 12, wherein determining if a substrate has been modified comprises performing SDS-PAGE of at least a portion of the combination of substrate and sample.

19. A method of detecting the presence of a protein phosphatase, said method comprising
providing at least two different protein phosphatase substrates, said substrates independently comprising a tag and at least one target site specific for a protein phosphatase,
wherein all target sites present on one substrate are specific for the same protein phosphatase, and
wherein each substrate differs in size from every other substrate or wherein each substrate that is specific for a protein phosphatase or group of phosphatases that share a common target site comprises a different tag from every substrate that is specific for a different protein phosphatase or group of phosphatases that share a common target site;
providing a sample that contains or is suspected of containing a protein phosphatase;
combining the substrate and sample for an amount of time sufficient for a protein phosphatase to modify a substrate; and
determining if a substrate has been modified,
wherein the presence of a modified substrate indicates the presence of at least one protein phosphatase that specifically modifies the substrate.

20. The method of claim 19, wherein the combining is performed in vitro.

21. The method of claim 19, wherein the combining is performed in vivo.

22. The method of claim 19, wherein each substrate is provided in an amount of about 0.1 μg to about 1.0 μg.

23. The method of claim 19, wherein each substrate is bound to a solid support.

24. The method of claim 19, wherein determining if a substrate has been modified comprises performing SDS-PAGE of at least a portion of the combination of substrate and sample.

25. A method of making a nucleic acid, said method comprising:
engineering a nucleic acid fragment encoding an amino acid sequence of interest;
synthesizing a double-stranded fragment having one end that is a competent endonuclease restriction cleavage site product and another end that is an incompetent endonuclease restriction cleavage site product, such that ligation of the competent site product to another competent site product produces a site that is recognized by an endonuclease, whereas ligation of the incompetent site product to a competent site product results in successful ligation, but creation of a site that is not recognized by the same endonuclease, thus permitting directional cloning of the fragment;
ligating the fragment to one or more other fragments encoding amino acid sequences or to one or more heterologous nucleic acids encoding or capable of expressing an amino acid sequence;
cleaving ligation products containing competent sites with the endonuclease;
optionally purifying one or more cleavage products; and
optionally, cloning the cleavage products into a vector permitting expression of the encoded amino acid sequence of the cleavage products.

26. A kit comprising at least two different protein kinase substrates, said substrates independently comprising a tag and at least one target site specific for a protein kinase or protein phosphatase,
wherein all target sites present on one substrate are specific for the same protein kinase or protein phosphatase,
wherein each substrate is specific for a different protein kinase or set of protein kinases that share a common target site, or specific for a different protein phosphatase or set of protein phosphatases that share a common target site, and
wherein each substrate differs in size from every other substrate or each substrate that is specific for a particular kinase or phosphatase has a different tag from all other substrates.

27. The kit of claim 26, wherein each of the different substrates are contained in different containers.

28. The kit of claim 26, further comprising a reaction buffer for phosphorylation of substrates by one or more protein kinases.

29. The kit of claim 26, wherein the substrates comprise sequences specific for CHK, CK1, CK2, EGFR, ERK, IKK, JNK, MAPK, JNK3, PKA, AMPK, DNA-PK, EF2K, FAK, GRK, PKC, CDK1, ATM, Abl, Src, or combinations of these.

30. The kit of claim 26, wherein the kit comprises substrates specific for EGFR, IKK, JNK3, and Src in a single container.


SEQUENCE LISTING (42 sequences)

SEQ ID NO: 1 - DNA, 36 bp, Artificial Sequence
  Description of Artificial Sequence Synthetic primer
gcgcggatccggtggcagctggcgtaatagcgaaga

SEQ ID NO: 2 - DNA, 35 bp, Artificial Sequence
  Description of Artificial Sequence Synthetic primer
gcgcgaattcgcctttatgcagcaacgagacgtca

SEQ ID NO: 3 - DNA, 33 bp, Artificial Sequence
  Description of Artificial Sequence Synthetic primer
gcgcggatccggacaacgtcgtgactgggaaaa

SEQ ID NO: 4 - DNA, 30 bp, Artificial Sequence
  Description of Artificial Sequence Synthetic primer
gcgcgaattcaccattttcaatccgcacct

SEQ ID NO: 5 - DNA, 32 bp, Artificial Sequence
  Description of Artificial Sequence Synthetic primer
gcgcggatccggtcacgccatcccgcatctga

SEQ ID NO: 6 - DNA, 34 bp, Artificial Sequence
  Description of Artificial Sequence Synthetic primer
gcgcgaattcgccttgacaccagaccaactggta

SEQ ID NO: 7 - protein, 7 aa, Artificial Sequence
  Description of Artificial Sequence Synthetic peptide
AIYAAPF

SEQ ID NO: 8 - DNA, 27 bp, Artificial Sequence
  Description of Artificial Sequence Synthetic oligonucleotide
aattcgcgatttatgcggcgccgtttc

SEQ ID NO: 9 - DNA, 27 bp, Artificial Sequence
  Description of Artificial Sequence Synthetic oligonucleotide
aattgaaacggcgccgcataaatcgcg

SEQ ID NO: 10 - protein, 22 aa, Artificial Sequence
  Description of Artificial Sequence Synthetic peptide
DQEAKVSASGLYRSPSMPENLN

SEQ ID NO: 11 - DNA, 72 bp, Artificial Sequence
  Description of Artificial Sequence Synthetic oligonucleotide
aattcgatcaggaagcgaaagtgagtgcgagtggtctgtatcgttctccgtctatgccggaaaacctgaacc

SEQ ID NO: 12 - DNA, 72 bp, Artificial Sequence
  Description of Artificial Sequence Synthetic oligonucleotide
aattggttcaggttttccggcatagacggagaacgatacagaccactcgcactcactttcgcttcctgatcg

SEQ ID NO: 13 - protein, 15 aa, Artificial Sequence
  Description of Artificial Sequence Synthetic peptide
DLHDDEEDEAMSITA

SEQ ID NO: 14 - DNA, 51 bp, Artificial Sequence
  Description of Artificial Sequence Synthetic oligonucleotide
aattcgatctgcatgatgatgaagaagacgaggcgatgtctatcaccgcac

SEQ ID NO: 15 - DNA, 51 bp, Artificial Sequence
  Description of Artificial Sequence Synthetic oligonucleotide
aattgtgcggtgatagacatcgcctcgtcttcttcatcatcatgcagatcg

SEQ ID NO: 16 - protein, 9 aa, Artificial Sequence
  Description of Artificial Sequence Synthetic peptide
RRDDDSDDD

SEQ ID NO: 17 - DNA, 33 bp, Artificial Sequence
  Description of Artificial Sequence Synthetic oligonucleotide
aattccgtcgcgatgacgatagcgatgatgacc

SEQ ID NO: 18 - DNA, 33 bp, Artificial Sequence
  Description of Artificial Sequence Synthetic oligonucleotide
aattggtcatcatcgctatcgtcatcgcgacgg

SEQ ID NO: 19 - protein, 18 aa, Artificial Sequence
  Description of Artificial Sequence Synthetic peptide
GGGEETQTQDQPMEEEEV

SEQ ID NO: 20 - DNA, 60 bp, Artificial Sequence
  Description of Artificial Sequence Synthetic oligonucleotide
aattcggcggtggcgaagaaacccagacccaggatcagccgatggaagaggaagaagttc

SEQ ID NO: 21 - DNA, 60 bp, Artificial Sequence
  Description of Artificial Sequence Synthetic oligonucleotide
aattgaacttcttcctcttccatcggctgatcctgggtctgggtttcttcgccaccgccg

SEQ ID NO: 22 - protein, 11 aa, Artificial Sequence
  Description of Artificial Sequence Synthetic peptide
AENAEYLRVAP

SEQ ID NO: 23 - DNA, 39 bp, Artificial Sequence
  Description of Artificial Sequence Synthetic oligonucleotide
aattcgcagaaaacgcagaatatctgcgtgtggcaccgc

SEQ ID NO: 24 - DNA, 39 bp, Artificial Sequence
  Description of Artificial Sequence Synthetic oligonucleotide
aattgcggtgccacacgcagatattctgcgttttctgcg

SEQ ID NO: 25 - protein, 10 aa, Artificial Sequence
  Description of Artificial Sequence Synthetic peptide
KQAEAVTSPR

SEQ ID NO: 26 - DNA, 36 bp, Artificial Sequence
  Description of Artificial Sequence Synthetic oligonucleotide
aattcaaacaggcagaagcagtgacctctccgcgtc

SEQ ID NO: 27 - DNA, 36 bp, Artificial Sequence
  Description of Artificial Sequence Synthetic oligonucleotide
aattgacgcggagaggtcactgcttctgcctgtttg

SEQ ID NO: 28 - protein, 7 aa, Artificial Sequence
  Description of Artificial Sequence Synthetic peptide
DSGLDSM

SEQ ID NO: 29 - DNA, 27 bp, Artificial Sequence
  Description of Artificial Sequence Synthetic oligonucleotide
aattcgatagcggtctggattccatgc

SEQ ID NO: 30 - DNA, 27 bp, Artificial Sequence
  Description of Artificial Sequence Synthetic oligonucleotide
aattgcatggaatccagaccgctatcg

SEQ ID NO: 31 - protein, 16 aa, Artificial Sequence
  Description of Artificial Sequence Synthetic peptide
ELVEPLTPSGEAPNQA

SEQ ID NO: 32 - DNA, 54 bp, Artificial Sequence
  Description of Artificial Sequence Synthetic oligonucleotide
aattcgaactggtggaaccgctgactccaagcggcgaagcaccgaaccaggcac

SEQ ID NO: 33 - DNA, 54 bp, Artificial Sequence
  Description of Artificial Sequence Synthetic oligonucleotide
aattgtgcctggttcggtgcttcgccgcttggagtcagcggttccaccagttcg

SEQ ID NO: 34 - protein, 21 aa, Artificial Sequence
  Description of Artificial Sequence Synthetic peptide
AGGTGGSLRRASLPGTGGSEL

SEQ ID NO: 35 - DNA, 69 bp, Artificial Sequence
  Description of Artificial Sequence Synthetic oligonucleotide
aattcgcaggcggtaccggcggcagcctgcgtcgcgcgagtctgccgggtaccggtggcagcgaactgc

SEQ ID NO: 36 - DNA, 69 bp, Artificial Sequence
  Description of Artificial Sequence Synthetic oligonucleotide
aattgcagttcgctgccaccggtacccggcagactcgcgcgacgcaggctgccgccggtaccgcctgcg

SEQ ID NO: 37 - protein, 20 aa, Artificial Sequence
  Description of Artificial Sequence Synthetic peptide
HMRSAMSGLHLQFHMRSAMS

SEQ ID NO: 38 - DNA, 116 bp, Artificial Sequence
  Description of Artificial Sequence Synthetic oligonucleotide
aattccatatgcgcagtgcaatgtctggtctgcatctgcaattccatatgcgcagtgcaatgtcgnnnnnnnnnnnnnnnnnnnnnnnnnnnnnnnnnnnnnnnnnnnnnnnnnnc

SEQ ID NO: 39 - DNA, 116 bp, Artificial Sequence
  Description of Artificial Sequence Synthetic oligonucleotide
aattgnnnnnnnnnnnnnnnnnnnnnnnnnnnnnnnnnnnnnnnnnnnnnnnnnncgacattgcactgcgcatatggaattgcagatgcagaccagacattgcactgcgcatatgg

SEQ ID NO: 40 - protein, 7 aa, Artificial Sequence
  Description of Artificial Sequence Synthetic peptide
ETYGEFG

SEQ ID NO: 41 - DNA, 27 bp, Artificial Sequence
  Description of Artificial Sequence Synthetic oligonucleotide
aattcgaaacctatggcgaatttggcc

SEQ ID NO: 42 - DNA, 27 bp, Artificial Sequence
  Description of Artificial Sequence Synthetic oligonucleotide
aattggccaaattcgccataggtttcg